(19) United States
(12) Patent Application Publication
(10) Pub. No.: US 2022/0226448 A1
(43) Pub. Date: Jul. 21, 2022

(54) ONE-STEP ARTIFICIAL ANTIGEN PRESENTING CELL-BASED VACCINES

(71) Applicant: BAYLOR RESEARCH INSTITUTE, Dallas, TX (US)

(72) Inventors: Botond Z. IGYÁRTÓ, Dallas, TX (US); Qingtai SU, Dallas, TX (US)

(73) Assignee: BAYLOR RESEARCH INSTITUTE, Dallas, TX (US)

(21) Appl. No.: 17/595,588

(22) PCT Filed: May 22, 2020
(86) PCT No.: PCT/US2020/034182

§ 371 Date: Nov. 19, 2021

Related U.S. Application Data
(60) Provisional application No. 62/851,537, filed on May 22, 2019.

(51) Int. Cl. A61K 39/00; A61K 39/39; A61P 35/00

(52) U.S. Cl. CPC A61K 39/0011; A61K 39/39; A61P 35/00; A61K 2039/55533; A61K 2039/6018; A61K 2039/55527; A61K 2039/55522; A61K 2039/5158; A61K 2039/55538

(57) ABSTRACT

Methods and compositions using artificial antigen presenting cells in immunotherapy. The artificial antigen presenting cells comprise substrate particles bound to endogenous MHC-peptide complexes obtained from one or more cells. Methods include administering the artificial antigen presenting cells to a patient to activate antigen-specific T cells.

### BACKGROUND OF THE INVENTION

### I. Field of the Invention

This invention relates to the field of immunotherapy. More specifically, the invention relates to artificial antigen presenting cells, which may be made by directly capturing MHC-peptide complexes from cell lysates, and therapeutic methods that use such artificial antigen presenting cells.

### II. Background

Dendritic cells (DCs), as professional antigen presenting cells (APCs), coordinate every aspect of immunity (Fu and Jiang, 2018; Merad et al., 2013; Sallusto and Lanzavecchia, 2002). DCs stimulate and train other immune cells, such as T cells, of the adaptive immune system by presenting peptide epitopes derived from antigens (self, cancer neoantigens, foreign, etc.) on their MHC, and providing co-stimulatory signals in membrane bound and secreted forms (cytokines) (Fu and Jiang, 2018; Merad et al., 2013; Sallusto and Lanzavecchia, 2002). These three signals train the T cells to recognize, destroy or tolerate the cells that carry these antigens. Thus, not surprisingly, the DCs are the main target of immunotherapies. Two main strategies are used to exploit DCs for cancer therapy (Palucka and Banchereau, 2013). One of them requires isolation of patient monocytes from blood and complex in vitro manipulation that involves differentiation and maturation into DCs using cytokine and adjuvant cocktails and pulsing with the chosen antigen(s)/cell lysates, followed by reinfusion into the patient. An alternative approach uses antibody as a carrier to deliver antigens to DCs in vivo. However, both of these approaches have shown only limited success rate, mainly because the DCs' function is highly dependent on the tumor environment, which is often inhibitory (Fu and Jiang, 2018; Palucka and Banchereau, 2013). Antigen-pulsed DCs can also be used to generate and activate a population of target antigen-specific T cells ex vivo, which population can then be transferred back into the patient, which is known as adoptive cell transfer. Preparation of DCs that are loaded with antigen and properly differentiated and activated is a difficult and time-consuming task that can limit T cell based therapy.

One approach to overcoming these obstacles that has recently been developed are artificial APCs (aAPCs), which are platforms for T cell activation and expansion. aAPCs could be a viable alternative to live DCs since they do not react to the environmental cues (Han et al., 2011; Lu et al., 2008; Oelke et al., 2003; Reddy et al., 2006; Shao et al., 2018; Steenblock and Fahmy, 2008; Ugel et al., 2009; Wang et al., 2017). aAPCs typically include a substrate, such as a polymer bead or lipid bilayer, on the surface of which are MHC I-peptide antigen complexes that can activate T cells by binding a complementary T cell receptor (TCR). aAPCs also may include co-stimulatory molecules that bind and activate such T cell membrane proteins as CD28 or CD40L. aAPCs have shown promise in generating anti-tumor T cell responses.

The production and wide use of artificial antigen presenting cells (aAPCs) in clinic as cancer immunotherapeutics has been hindered by the need to identify immunogenic cancer antigens expressed by a patient's tumor cells and to produce recombinant patient-specific major histocompatibility complexes (MHC) loaded with these peptides (Chaput et al., 2003; Laport et al., 2003; Mitchell et al., 2002). This process is time consuming and can significantly delay the administration of therapy. In addition, the recombinant HLA and peptides might not carry all the posttranslational modifications that would normally occur in vivo that could result in less effective TCR stimulation. Current aAPC treatment strategies are also limited in their efficacy because they typically target only one cancer antigen, whereas many tumors have a heterogeneous population of cells expressing different antigen repertoires. Therefore, targeting only a single cancer antigen may not be effective to eliminate the tumor.

### SUMMARY OF THE INVENTION

The inventors have developed an aAPC-based therapeutic strategy that overcomes the problems discussed above. In some embodiments, the strategy involves creating aAPCs in one step by directly capturing peptide-MHC complexes from cell lysates, including lysates of cancer cells obtained from a patient. This results in an aAPC composition that includes multiple peptide-MHC complexes expressed by the cancer cells, which can then be used to generate a population of activated T cells targeting multiple cancer antigens. This strategy avoids the need to identify specific antigens expressed by the patient and to prepare recombinant MHC I-peptide complexes. Thus, the methods disclosed herein can reduce the time and cost of providing a T cell therapy and enhance the efficacy of such therapy. Methods disclosed herein can also use the one-step aAPC production strategy in therapies to combat autoimmune disorders.

Embodiments concern methods of producing activated antigen-specific cytotoxic T cells, methods of killing target cells, methods of treating a condition in a patient, methods of treating cancer in a patient, methods of making an artificial antigen presenting cell, methods of inducing immune tolerance to an antigen in a subject, methods of using a composition, compositions for use in a method of making a medicament, compositions for use in a method of treating a condition in a patient, compositions for use in a method of treating cancer in a patient, compositions comprising artificial antigen presenting cells, and/or pharmaceutical compositions.

Disclosed herein is a method of producing activated antigen-specific cytotoxic T cells, the method comprising contacting CD8+ T cells with a composition comprising substrate particles bound to endogenous MHC I-peptide complexes obtained from one or more cells. As used herein, “endogenous MHC I-peptide complexes” refers to peptide-bound class I major histocompatibility complexes produced by cells without the use of recombinant or synthetic nucleic acids or polypeptides. In some embodiments, the substrate particles in the composition are not bound to any recombinantly expressed MHC complexes. In some embodiments, the contacting causes activation and proliferation of CD8+ T cells specific for one or more MHC I-peptide complexes present in the MHC I-peptide complexes bound to the substrate particles. The cells from which the MHC I-peptide complexes are obtained may be human or non-human cells, and the MHC complexes may be human MHC complexes or may be non-human equivalents thereof. In some embodiments, the endogenous MHC I-peptide complexes are obtained from a lysate prepared from one or more cells. In some embodiments, the method further comprises coupling the substrate particles to the endogenous MHC I-peptide complexes obtained from the one or more cells by a method comprising: contacting a lysate of the one or more cells with substrate particles bound to a polypeptide capable of specifically binding MHC, wherein the lysate of the one or more cells contains endogenous MHC I-peptide complexes produced by the one or more cells. In some embodiments, the endogenous MHC I-peptide complexes comprise a plurality of different MHC I-peptide complexes. By “a plurality of different MHC I-peptide complexes,” it is meant that the MHC I-peptide complexes bound to the substrate particles include MHC complexes bound to peptide antigens having two or more different sequences. In some embodiments, at least 2, 3, 4, 5, 6, 7, 8, 9, 10, 20, 30, 40, 50, 100, 500, 1000, or 5000 or more different peptide sequences are bound to MHC complexes bound to the substrate particles, or any range derivable therein. In some embodiments, the plurality of different endogenous MHC I-peptide complexes represents a repertoire of the MHC I-peptide complexes expressed by the one or more cells. In some embodiments, the endogenous MHC I-peptide complexes comprise peptides from one or more tumor-specific antigens.

The endogenous MHC I-peptide complexes bound to the substrate particles may be obtained from a single cell or from multiple cells. In some embodiments, the endogenous MHC I-peptide complexes are obtained from a single cell. In some embodiments, the method of producing activated antigen-specific cytotoxic T cells further comprises coupling the substrate particles to the endogenous MHC I-peptide complexes obtained from a single cell by a method comprising: contacting a lysate prepared from the single cell with substrate particles bound to a polypeptide capable of specifically binding MHC, wherein the lysate contains endogenous MHC I-peptide complexes produced by the cell. In some embodiments, the single cell is comprised within a continuous lipid membrane with a single substrate particle before it is lysed.

In some embodiments, the one or more cells comprise one or more cancer cells obtained from a patient. Such embodiments may be part of a method of treating cancer in the patient. In some embodiments, the one or more cancer cells obtained from the patient comprise a tumor biopsy. In some embodiments, the one or more cancer cells comprise a heterogeneous population of cancer cells. In some embodiments, the heterogeneous population of cancer cells comprises cancer cells having different antigen repertoires. In some embodiments, the endogenous MHC I-peptide complexes produced by the cancer cells include cancer antigens that may be effectively targeted by activated, antigen specific cytotoxic T cells. Thus, in some embodiments, contacting CD8+ T cells with substrate particles bound to endogenous MHC I-peptide complexes produced by the cancer cells leads to activation and proliferation of CD8+ T cells capable of killing cancer cells in the patient.

In some embodiments of the method of producing activated antigen-specific cytotoxic T cells, contacting the CD8+ T cells with the composition occurs in vivo. For example, in some embodiments, the CD8+ T cells are a patient's cells present within the patient's body at the time of the contacting. The contacting may occur as a result of administering the composition comprising the substrate particles bound to the endogenous MHC I-peptide complexes to the patient. In some embodiments, contacting the CD8+ T cells with the composition occurs ex vivo. In some embodiments, the CD8+ T cells have been removed from a patient before the contacting. In some embodiments, the method further comprises administering the CD8+ T cells to the patient after the contacting.

In some embodiments, the substrate particles comprise a polymer material, a magnetic material, or a lipid bilayer. The substrate particles may be, for, example spherical synthetic beads, which may comprise a polymer material and/or a magnetic material. Substrate particles comprising a lipid bilayer may also be cell-based, such as engineered K562 cells, or may be synthetic liposomes or other lipid vesicles. In some embodiments, the substrate particles are biodegradable. In some embodiments the biodegradable substrate particles are capable of biodegrading in the human body within 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 days of being placed in the human body, or any range derivable therein. In some embodiments, the particles in the composition are between about 0.5 and 500 μm, between about 1 and 20 μm in size, or between about 1 and 500 nm in size. As used herein, the size of a particle is measured as its largest dimension; thus, for spherical substrate particles, the size is measured as the diameter. In some embodiments, the mean size of the substrate particles in the composition is between about 0.5 and 500 μm, between about 1 and 20 μm, or between about 1 and 500 nm. In some embodiments, the mean size or median size (number distribution) of the substrate particles in the composition is at least about, at most about, or about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, 100, 125, 150, 175, 200, 225, 250, 275, 300, 325, 350, 375, 400, 425, 450, 475, or 500 μm, or between any two of these values, or is at least about, at most about, or about 1, 5, 10, 20, 30, 40, 50, 60, 70, 80, 90, 100, 125, 150, 175, 200, 225, 250, 275, 300, 325, 350, 375, 400, 425, 450, 475, or 500 nm, or between any two of these values. The substrate particles may take a variety of different shapes, including for example, spherical, ellipsoid, or rod-shaped.

The substrate particles can be bound to the endogenous MHC I-peptide complexes in a variety of ways, which can be chosen by a person of ordinary skill in the art based on the material chosen for the substrate particles. For example, in some embodiments, the endogenous MHC I-peptide complexes are bound to the substrate particles by a polypeptide capable of specifically binding MHC. In some embodiments, the polypeptide that specifically binds MHC comprises an MHC-binding antibody or functional fragment thereof, of which several are known in the art. In some embodiments, the polypeptide that specifically binds MHC comprises a viral or bacterial protein. In some embodiments, the polypeptide that specifically binds MHC comprises an in silico designed polypeptide that is synthesized in vitro.

In some embodiments, the substrate particles are additionally bound to a molecule that is capable of providing a co-stimulatory signal to the CD8+ T cells that helps induce activation and proliferation of antigen-specific T cells. In some embodiments, the substrate particles are additionally bound to one or more of the following T cell co-stimulatory molecules: CD80, CD86, OX-40L, 4-1BBL, CD70, ICOS-L, and/or GITR-L. Other co-stimulatory molecules known in the art may also be included, and any combination of 2, 3, 4, 5, 6, 7, 8, 9, 10 or more of the co-stimulatory molecules disclosed herein or known in the art may be used (or specifically excluded). In some embodiments, the particles are additionally bound to one or more polypeptides that specifically bind and activate one or more of the following T cell molecules: CD28, OX40, 4-1BB, CD27, ICOS, and/or GITR, including any combination of 2, 3, 4, 5, or 6 of these T cell molecules. In some embodiments, the one or more polypeptides that specifically bind and activate a T cell molecule comprises an antibody or functional fragment thereof.

In some embodiments, the substrate particles further comprise a T cell activating molecule, which in some embodiments is a cytokine. In some embodiments, the T cell activating molecule is embedded in the substrate particles. In some embodiments, the T cell activating molecule is capable of being released from the substrate particles into the media. In some embodiments, the released T cell activating molecule is released into the media and makes contact with the CD8+ T cells. In some embodiments, the T cell molecule is capable of being released from the substrate particles into the media only upon or after contacting the CD8+ T cells. In some embodiments, the T cell activating cytokine is selected from IL-2, IL-7, IL-12, IL-15, and/or IL-21. Other T cell activating cytokines known in the art may also be used, and any combination of 2, 3, 4, 5, 6, 7, 8, 9, 10 or more of the T cell activating cytokines disclosed herein or known in the art may be used. In some embodiments, the T cell activating molecule stimulates and/or activates the CD8+ T cells. In some embodiments, the substrate particles further comprise a T cell chemoattractant. In some embodiments, the T cell chemoattractant is selected from CXCL9, CXCL10, CXCL12 CXCL16, CCL3, CCL4, CCL19, and/or CCL21. Other chemoattractants known in the art may also be used, and any combination of 2, 3, 4, 5, 6, 7, 8, 9, 10 or more of the T cell activating cytokines disclosed herein or known in the art may be used. Though in some embodiments, one or more of these chemoattractants may be specifically excluded.

Also disclosed is a method of killing a target cell, the method comprising: (a) contacting CD8+ T cells with a with a composition comprising substrate particles bound to endogenous MHC I-peptide complexes obtained from one or more cells of the same type as the target cell, thereby creating activated, target antigen-specific CD8+ T cells; and (b) contacting the target cell with the activated, target antigen-specific CD8+ T cells, thereby killing the target cell. In some embodiments, a given cell and a target cell are determined to be the same type of cell based on a comparison of their mRNA expression profiles, miRNA expression profiles, proteomes, one or more cell surface antigens, genomic methylation profiles, or other epigenetic profiles. In some embodiments, a given cell and a target cell are determined to be the same type of cell based on a comparison of the morphological features or based on their being from the same tissue. In some embodiments, steps (a) and (b) occur in vivo. In some embodiments, step (a) comprises administering the composition to a patient to cause the contacting. In some embodiments, the endogenous MHC I-peptide complexes are obtained from a lysate prepared from the one or more cells. In some embodiments, the endogenous MHC I-peptide complexes comprise a plurality of different MHC I-peptide complexes.

Also disclosed is a method of treating a condition in a patient comprising administering to the patient a composition comprising substrate particles bound to endogenous MHC I-peptide complexes obtained from one or more cells of the same type as a target cell, wherein the target cell is killed, and wherein killing the target cell treats the condition. In some embodiments, the condition is cancer. In some embodiments, the one or more cells were obtained from the patient. In some embodiments, the target cell is a cancer cell and the one or more cells from which the endogenous MHC I-peptide complexes are obtained are cancer cells of the same type of cancer. In some embodiments, the target cell is a tumor cell and the one or more cells from which the endogenous MHC I-peptide complexes are obtained are cells from the same tumor in which the target cell was present before being killed or from a metastasis of that tumor. In some embodiments, the target cell is a leukemia, lymphoma, or myeloma cell in a patient's body, and the one or more cells from which the endogenous MHC I-peptide complexes are obtained are of the same type of leukemia, lymphoma, or myeloma and were obtained from the patient's blood or bone marrow.

Also disclosed is a method of treating cancer in a patient comprising: (a) preparing a solution comprising endogenous MHC I-peptide complexes obtained from one or more cancer cells; (b) contacting the solution with substrate particles bound to a polypeptide that specifically binds MHC to pull down the endogenous MHC I-peptide complexes; and (c) administering the substrate particles produced in step (b) to the patient. In some embodiments, the solution is a lysate of the one or more cancer cells. In some embodiments, the lysate is prepared from a single cancer cell. In some embodiments, the lysate is prepared from a tissue sample obtained from the patient.

Also disclosed is a method of making an artificial antigen presenting cell, the method comprising contacting a lysate prepared from one or more cells with substrate particles bound to a polypeptide that is capable of specifically binding MHC, thereby causing the substrate particles to become bound to a plurality of different endogenous MHC I-peptide complexes expressed by the one or more cells. In some embodiments, the lysate is prepared from a single cell. In some embodiments, the substrate particles are further bound to one or more of the following T-cell co-stimulatory molecules: CD80, CD86, OX-40L, 4-1BBL, CD70, ICOS-L, and/or GITR-L, or any combination thereof. In some embodiments, the substrate particles are further bound to one or more polypeptides that specifically bind and activate one or more of the following T cell molecules: CD28, OX40, 4-1BB, CD27, ICOS, and/or GITR, or any combination thereof. In some embodiments, the substrate particles comprise a polymer material, a magnetic material, or a lipid bilayer. In some embodiments, the substrate particles are biodegradable.

Also disclosed is a composition comprising substrate particles bound to (a) a plurality of different endogenous MHC I-peptide complexes, wherein the MHC I-peptide complexes are bound to the substrate particles via a polypeptide that specifically binds MHC; and (b) one or more of the following molecules: (i) a T-cell costimulatory molecule selected from CD80, CD86, OX-40L, 4-1BBL, CD70, ICOS-L, and/or GITR-L; or (ii) one or more polypeptides that specifically bind and activate one or more of the following T cell molecules: CD28, OX40, 4-1BB, CD27, ICOS, and/or GITR. In some embodiments, the substrate particles further comprise a signaling molecule that can be released from the substrate particles and modulate T cell activity. In some embodiments, the signaling molecule comprises a T cell activating cytokine selected from IL-2, IL-7, IL-12, IL-15, and/or IL-21 or a T cell chemoattractant selected from one or more of CXCL9, CXCL10, CXCL12, CXCL16, CCL3, CCL4, CCL19, and/or CCL21. In some embodiments, the substrate particles comprise a polymer material, a magnetic material, or a lipid bilayer. In some embodiments, the substrate particles are biodegradable. In some embodiments, the plurality of different endogenous MHC I-peptide complexes represents a repertoire of MHC I-peptide complexes expressed by one or more cells.

Also disclosed is a method of inducing immune tolerance to an antigen in a subject, the method comprising: (a) obtaining artificial antigen presenting cells comprising substrate particles bound to: (i) endogenous MHC II complexes bound to peptides of the antigen, wherein the endogenous MHC II complexes were obtained from one or more cells containing the autoantigen; and (ii) one or more inhibitory ligands selected from B7.1wa, CTLA-4 binding proteins, PD-L1, HVEM, PDL-2, B7-H3, B7-H4, OX-2, TGF-beta1, IL-10, IL-4, natural, recombinant and artificial ligands for CTLA-4, PD-1, OX-2 receptor, B7-H3 receptor, B7-H4 receptor and BTLA, natural and recombinant anti-CTLA-4 agonistic antibody, anti-PD-1 agonistic antibody, anti BTLA agonistic Antibody Anti-B7-H3 receptor agonistic antibody, Anti-B7-H4 receptor antibody, anti-CD28 antagonistic antibody, and/or anti-ICOS antagonistic antibody, including any combination of 2, 3, 4, 5, 6, 7, 8, 9, or 10 of these ligands.

Also disclosed is a method of inducing immune tolerance to an antigen in a subject, the method comprising: (a) obtaining artificial antigen presenting cells comprising substrate particles bound to: (i) endogenous MHC II complexes bound to peptides of the antigen, wherein the endogenous MHC II complexes were obtained from one or more cells containing the autoantigen; and (ii) one or more inhibitory ligands selected from B7.1wa, CTLA-4 binding proteins, PD-L1, HVEM, PDL-2, B7-H3, B7-H4, OX-2, TGF-beta1, IL-10, IL-4, natural, recombinant and artificial ligands for CTLA-4, PD-1, OX-2 receptor, B7-H3 receptor, B7-H4 receptor and BTLA, natural and recombinant anti-CTLA-4 agonistic antibody, anti-PD-1 agonistic antibody, anti BTLA agonistic Antibody Anti-B7-H3 receptor agonistic antibody, Anti-B7-H4 receptor antibody, anti-CD28 antagonistic antibody, and/or anti-ICOS antagonistic antibody; (b) ex vivo, contacting regulatory T cells specific for the antigen with the artificial antigen presenting cells; and (c) administering the regulatory T cells to the subject.

Also disclosed is a method of treating a condition in a patient, the method comprising: (a) obtaining cells from the patient; (b) preparing a lysate from the cells, wherein the lysate comprises endogenous MHC-peptide complexes produced by the cells; (c) contacting the lysate with substrate particles bound to an anti-MHC antibody or functional fragment thereof, thereby generating aAPCs comprising the substrate particles bound to the endogenous MHC-peptide complexes; (d) administering the aAPCs to the patient. In some embodiments, the condition is cancer and the cells obtained from the patient comprise cancer cells.

Also disclosed is a method of treating a condition in a patient, the method comprising administering aAPCs to the patient, wherein the aAPCs comprise substrate particles bound to endogenous MHC-peptide complexes, wherein the aAPCs have been prepared by contacting a lysate prepared from cells obtained from the patient with the substrate particles, and wherein the substrate particles are bound to a polypeptide capable of specifically binding endogenous MHC I-peptide complexes in the lysate.

As used herein, “MHC I” refers to human major histocompatibility class I complex and equivalent complexes in non-human animals, unless the context in which “MHC I” appears indicates that it is meant to refer only to the human complex or only to non-human equivalents of the human complex.

As used herein, “MHC II” refers to human major histocompatibility class II complex and equivalent complexes in non-human animals, unless the context in which “MHC II” appears indicates that it is meant to refer only to the human complex or only to non-human equivalents of the human complex.

The term “lysate” as used herein refers to the composition obtained when cells are lysed and optionally the cellular debris (e.g., cellular membranes) is removed. “Lysate” does not include solutions of proteins purified from such a composition.

Throughout this application, the term “about” is used to indicate that a value includes the inherent variation of error for the measurement or quantitation method.

The use of the word “a” or “an” when used in conjunction with the term “comprising” may mean “one,” but it is also consistent with the meaning of “one or more,” “at least one,” and “one or more than one.”

The phrase “and/or” means “and” or “or”. To illustrate, A, B, and/or C includes: A alone, B alone, C alone, a combination of A and B, a combination of A and C, a combination of B and C, or a combination of A, B, and C. In other words, “and/or” operates as an inclusive or.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. Compositions and methods “consisting essentially of” any of the ingredients or steps disclosed limits the scope of the claim to the specified materials or steps which do not materially affect the basic and novel characteristic of the claimed invention.

It is contemplated that any embodiment discussed in this specification can be implemented with respect to any method or composition of the invention, and vice versa. Any features or methods described in the context of one embodiment may be incorporated into any of the other disclosed embodiments. Furthermore, compositions of the invention can be used to achieve methods of the invention, and method of the invention can be performed using compositions of the invention.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1—Graphical representation of one-step aAPC generation. Tumor cell lysates are incubated with affinity beads that capture the peptide-MHC repertoire of the cancer cells. The beads are then used to activate cancer antigen-specific T cell clones that will ultimately kill the tumor cells.

FIGS. 2A-C—aAPCs loaded with Kb:SIINFEKL can prime OT-I T cells. (A) Experimental flow: 293T cells were transfected with OVA and/or Kb-Ctag coding plasmids. After two days of culture the cells were lysed and the MHC-I complexes captured using affinity beads targeting the Ctag motif. The capture of MHC-I and MHC-I complexed with SIINFEKL were determined with the help of flow cytometry (bottom) using antibody that recognizes SIINFEKL bound to Kb. (B) OT-I cells were stimulated in vitro with SIINFEKL peptide, Kb/OVA aAPCs, control aAPCs or Kb/OVA aAPCs in the presence of Y3 antibody, and pictures taken two days later (right). Arrows point to some of the activated OT-I cell clusters induced by the stimulation. One representative experiment out of three is shown. Three samples/group, except SIINFEKL that served the role of positive control. (C) OT-I cells were stimulated with either Kb/OVA aAPCs or control aAPCs for 2 days and assessed by flow cytometry for activation (CD44) and proliferation (CTV dilution). One representative experiment out of three is shown. n=3. Unpaired t-test. *p<0.05; ***p<0.001.

FIGS. 3A-C—aAPC-activated OT-I T cells can kill tumor cells in vitro. (A) Experimental flow. OT-I cells were stimulated with SIINFEKL, control aAPC, or Kb/OVA aAPCs for 6 days. (B) Before their use for experiments the OT-I T cells' cytotoxic phenotype was confirmed by flow cytometry. Unpaired t-test, **p<0.01, ***p<0.001. (C) The OT-I cells were then mixed with B16-OVA (target cells) and B16 WT (control cells) cells and cultured for one day. The killing of target cells was determined by flow cytometer and shown as relative percentage (right). One representative experiment out of two is shown, n=3. One-way ANOVA. ****p<0.0001.

FIGS. 4A-C—aAPC-activated OT-I T cells can kill tumor cells in vivo. (A) Experimental flow. OT-I cells were stimulated with SIINFEKL, control aAPCs, or Kb/OVA aAPCss for 5 days. To determine the in vivo tumor killing potency of the aAPC-stimulated OT-I cells, the OT-I cells were transferred into B16-OVA tumor bearing mice and the tumor growth (B) (two-way ANOVA; Tukey's test ***p<0.001) and the survival (C) monitored as depicted (Long-rank, Mantel-Cox test, *p<0.05). Data from two experiments were combined. n=6-8 mice/group. In (b), the curve labeled 102 represents control aAPC, the curve labeled 104 represents SIINFEKL, and the curve labeled 106 represents KB/OVA aAPC. In (c), the curve labeled 108 represents control aAPC, the curve labeled 110 (which stays at 100% survival to the end of the experiment) represents Kb/OVA aAPC, and the curve labeled 112 represents SIINFEKL.

FIGS. 5A-C—aAPCs generated using B16F10 cells activated T cells from tumor-bearing mice. (A) Experimental flow for data presented on FIGS. 5B-C. B16F10 cells were transfected with Kb-Ctag plasmid. Two days later the cells were lysed and the MHC-I repertoire captured using affinity beads. The successful capture of the MHC-Is was confirmed with flow cytometry (B). The aAPCs were then used to stimulate T cells isolated from tumor bearing mice's spleen for six days. The induction of CD8 T cells with cytotoxic phenotype, characterized by IFNγ production, was confirmed using flow cytometry (C). One representative experiment out of two is shown. n=3, except unstimulated n=1. Unpaired t test. **p<0.01.

### DETAILED DESCRIPTION OF THE INVENTION

Compositions and methods disclosed herein address the need for artificial antigen-presenting cells that can be effectively used for treatment of various diseases and that can be produced in good time and with reduced expense. Below, embodiments of these compositions and methods are described in greater detail.

### I. Definitions

The terms “protein,” “polypeptide,” and “peptide” are used interchangeably herein when referring to a gene product. When referred to in context of a MHC I-peptide complex or MHC II-peptide complex, “peptide” refers to a portion of a protein that is bound to the indicated MHC molecule's peptide binding groove.

A “tumor-specific antigen” is defined herein as an antigen that is unique to tumor cells and does not occur in or on other cells in the body.

“Homology,” or “identity” refers to sequence similarity between two peptides or between two nucleic acid molecules. Identity can be determined by comparing a position in each sequence which may be aligned for purposes of comparison. When a position in the compared sequence is occupied by the same base or amino acid, then the molecules share sequence identity at that position. A degree of identity between sequences is a function of the number of matching or homologous positions shared by the sequences. An “unrelated” or “non-homologous” sequence shares less than 60% identity, less than 50% identity, less than 40% identity, less than 30% identity, or less than 25% identity, with one of the sequences of the current disclosure.

The terms “amino portion,” “N-terminus,” “amino terminus,” and the like as used herein are used to refer to order of the regions of the polypeptide. Furthermore, when something is N-terminal to a region it is not necessarily at the terminus (or end) of the entire polypeptide, but just at the N-terminus of the region or domain. Similarly, the terms “carboxy portion,” “C-terminus,” “carboxy terminus,” and the like as used herein is used to refer to order of the regions of the polypeptide, and when something is C-terminal to a region it is not necessarily at the terminus (or end) of the entire polypeptide, but just at the C-terminus of the region or domain.

The terms “polynucleotide,” “nucleic acid,” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, ribozymes, cDNA, dsRNA, siRNA, miRNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any embodiment of this invention that is a polynucleotide encompasses both the double-stranded form and each of two complementary single-stranded forms known or predicted to make up the double-stranded form.

Cells are “substantially free” of certain reagents or elements, such as serum, signaling inhibitors, animal components or feeder cells, exogenous genetic elements or vector elements, as used herein, when they have less than 10% of the element(s), and are “essentially free” of certain reagents or elements when they have less than 1% of the element(s). However, even more desirable are cell populations wherein less than 0.5% or less than 0.1% of the total cell population comprise exogenous genetic elements or vector elements.

A culture, matrix or medium are “essentially free” of certain reagents or elements, such as serum, signaling inhibitors, animal components or feeder cells, when the culture, matrix or medium respectively have a level of these reagents lower than a detectable level using conventional detection methods known to a person of ordinary skill in the art or these agents have not been extrinsically added to the culture, matrix or medium. The serum-free medium may be essentially free of serum.

As used herein, the terms “treatment,” “treating,” and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment,” as used herein, covers any treatment of a disease in a mammal, e.g., in a human, and includes: (a) preventing the disease from occurring in a subject which may be predisposed to the disease but has not yet been diagnosed as having it; (b) inhibiting the disease, i.e., arresting its development; and (c) relieving the disease, i.e., causing regression of the disease.

In some embodiments, the methods are useful for reducing the size and/or cell number of a tumor. In some embodiments, the method of the disclosure are useful for inhibiting the growth of tumors, such as solid tumors, in a subject. In some embodiments, the methods are useful for reducing the number of cancerous cells in a subject, which cancerous cells may include blood cells, for example.

The term “antibody” includes monoclonal antibodies, polyclonal antibodies, dimers, multimers, multispecific antibodies and antibody fragments that may be human, mouse, humanized, chimeric, or derived from another species. A “monoclonal antibody” is an antibody obtained from a population of substantially homogeneous antibodies that is being directed against a specific antigenic site.

“Antibody or functional fragment thereof” means an immunoglobulin molecule that specifically binds to, or is immunologically reactive with a particular antigen or epitope, and includes both polyclonal and monoclonal antibodies. The term “antibody” includes genetically engineered or otherwise modified forms of immunoglobulins, such as intrabodies, peptibodies, chimeric antibodies, fully human antibodies, humanized antibodies, and heteroconjugate antibodies (e.g., bispecific antibodies, diabodies, triabodies, and tetrabodies). The term “functional antibody fragment” includes antigen binding fragments of antibodies, including e.g., Fab′, F(ab)₂, Fab, Fv, rlgG, and scFv fragments. The term scFv refers to a single chain Fv antibody in which the variable domains of the heavy chain and of the light chain of a traditional two chain antibody have been joined to form one chain. In any embodiments disclosed herein in which an antibody is described, it is contemplated that an antibody or functional fragment thereof may be used.

The terms “bind,” “binding,” or “bound” refer to a direct association between two molecules, due to, for example, covalent, electrostatic, hydrophobic, and ionic and/or hydrogen-bond interactions, including interactions such as salt bridges and water bridges. As used herein, a protein may be “bound” to a substrate particle even if it does not directly contact the substrate particle, but is instead bound indirectly through one or more intermediate molecules. For example, in an aAPC having a substrate particle bound directly to an anti-MHC antibody which is in turn bound directly to an MHC I-peptide complex, the MHC I-peptide complex is “bound” to the substrate particle, even though the binding is indirect via the MHC I-peptide complex.

A “therapeutically effective amount” or “efficacious amount” refers to the amount of an agent, or combined amounts of two agents, that, when administered to a mammal or other subject for treating a disease, is sufficient to effect such treatment for the disease. The “therapeutically effective amount” will vary depending on the agent(s), the disease and its severity and the age, weight, etc., of the subject to be treated.

### II. Artificial Antigen Presenting Cells

Embodiments of the compositions and methods disclosed herein include artificial antigen presenting cells comprising substrate particles that are bound to or that include certain proteins. Various materials suitable for use in substrate particles are known in the art.

A. Materials for Substrate Particles

In some embodiments, the substrate particles are biodegradable. Examples of biodegradable substrate materials include, but are not limited to, biodegradable polymers such as polylactide, poly(lactic acid-co-glycolic acid), poly(dioxanone), poly(trimethylene carbonate) copolymer; poly(caprolactone) homopolymer, polyanhydride, polyorthoester, polyphosphazene, poly(caprolactone) copolymer, any polymeric substances based on polylactide (PLA), polyglycolide (PGA), polycaprolactone (PCL), Poly(ethylene oxide), Poly-alginate, PEO, Poly((lactide-co-ethyleneglycol)-co-ethyloxyphosphate), Poly(LAEG-EOP), Poly(1,4-bis(hydroxyethyl)terephthalate-co-ethyloxyphosphate), Poly(BHET-EOP), Poly(1,4-bis(hydroxyethyl)terephthalate-alt-ethyloxyphosphate)-co-1,4-bis(hydroxyethyl)terephthalate-co-terephthalate), Poly(BHET-EOP/TC, 80/20), and PMMA (Polymethylmethacrylate). Biodegradable polymers may include, for example, synthetic polymers that degrade by hydrolysis such as poly(hydroxy acids), such as polymers and copolymers of lactic acid and glycolic acid, polyanhydrides, poly(ortho)esters, polyesters, polyurethanes, poly(butic acid), poly(valeric acid), poly(caprolactone), poly(hydroxyalkanoates), and poly(lactide-co-caprolactone). Examples of preferred biodegradable polymers include synthetic polymers that degrade by hydrolysis such as poly(hydroxy acids), such as polymers and copolymers of lactic acid and glycolic acid, polyanhydrides, poly(ortho)esters, polyesters, polyurethanes, poly(butic acid), poly(valeric acid), poly(caprolactone), poly(hydroxyalkanoates), and poly(lactide-co-caprolactone). Other biodegradable substrate components include, but are not limited to, modified poly(saccharide)s, e.g., starch, cellulose, and chitosan; proteins (e.g., collagen, albumin, gelatin, elastin, silk fibroin), zein and other prolamines and hydrophobic proteins, lipid microspheres (e.g., prepared using lecithin and vegetable oils; and beta-estradiol microsphere). Some biodegradable materials that may be used for substrate particles include those degrade either by enzymatic hydrolysis or exposure to water in vivo, by surface or bulk erosion. Liposomes are also contemplated, wherein they can be modified to be bound to proteins on their surface. For example, liposomes carrying protein A, Protein L, protein G, Protein A/G, streptavidin, avidin, extravidin, biotin, antibodies, can be used to bind other proteins on their surface.

In some embodiments, non-biodegradable polymers can be used, especially hydrophobic polymers. Examples of preferred non-biodegradable polymers include ethylene vinyl acetate, poly(meth) acrylic acid, copolymers of maleic anhydride with other unsaturated polymerizable monomers, poly(butadiene maleic anhydride), polyamides, copolymers and mixtures thereof, and dextran, cellulose and derivatives thereof.

Other suitable biodegradable and non-biodegradable polymers include, but are not limited to, polyanhydrides, polyamides, polycarbonates, polyalkylenes, polyalkylene oxides such as polyethylene glycol, polyalkylene terepthalates such as poly(ethylene terephthalate), polyvinyl alcohols, polyvinyl ethers, polyvinyl esters, polyethylene, polypropylene, poly(vinyl acetate), poly vinyl chloride, polystyrene, polyvinyl halides, polyvinylpyrrolidone, polymers of acrylic and methacrylic esters, polysiloxanes, polyurethanes and copolymers thereof, modified celluloses, alkyl cellulose, hydroxyalkyl celluloses, cellulose ethers, cellulose esters, nitro celluloses, cellulose acetate, cellulose propionate, cellulose acetate butyrate, cellulose acetate phthalate, carboxyethyl cellulose, cellulose triacetate, cellulose sulfate sodium salt, and polyacrylates such as poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenyl methacrylate), poly(methyl acrylate), poly(isopropyl acrylate), poly(isobutyl acrylate), poly(octadecyl acrylate), agarose, and aldehyde activated agarose. The polymer may be a bioadhesive polymer that is hydrophilic or hydrophobic. Hydrophilic polymers include CARBOPOL™ (a high molecular weight, crosslinked, acrylic acid-based polymers manufactured by NOVEON™) polycarbophil, cellulose esters, and dextran.

The foregoing materials may be used alone, as physical mixtures (blends), or as co-polymers. In a preferred embodiment, the aAPCs are formed of polymers fabricated from polylactides (PLA) and copolymers of lactide and glycolide (PLGA). These have established commercial use in humans and have a long safety record (Jiang, et al., Adv. Drug Deliv. Rev., 57(3):391-410); Aguado and Lambert, Immunobiology, 184(2-3):113-25 (1992); Bramwell, et al., Adv. Drug Deliv. Rev., 57(9):1247-65 (2005)).

Rate controlling polymers may be included in the polymer matrix or in the coating on the formulation. Examples of rate controlling polymers that may be used are hydroxypropylmethylcellulose (HPMC) with viscosities of either 5, 50, 100 or 4000 cps or blends of the different viscosities, ethylcellulose, methylmethacrylates, such as EUDRAGIT® RS100, EUDRAGIT® RL100, EUDRAGIT® NE 30D (supplied by Rohm America). Gastrosoluble polymers, such as EUDRAGIT® E100 or enteric polymers such as EUDRAGIT® L100-55D, L100 and 5100 may be blended with rate controlling polymers to achieve pH dependent release kinetics. Other hydrophilic polymers such as alginate, polyethylene oxide, carboxymethylcellulose, and hydroxyethylcellulose may be used as rate controlling polymers.

These polymers can be obtained from sources such as Sigma Chemical Co., St. Louis, Mo.; Polysciences, Warrenton, Pa.; Aldrich, Milwaukee, Wis.; Fluka, Ronkonkoma, N.Y.; and BioRad, Richmond, Calif., or can be synthesized from monomers obtained from these or other suppliers using standard techniques.

In some embodiments, the substrate particles comprise magnetic or paramagnetic material. Beads that can be coated with or bound by coupling agents and/or affinity tags are known in the art, including, for example, Dynabeads™, Macs Microbeads™, and other materials.

B. Binding to Substrate Particles

The external surface of polymeric substrate particles may be modified by conjugating to, or incorporating into, the surface of the microparticle a coupling agent or ligand. Such coupling agents or ligands may be present on the surface of the substrate particle at a high density. Molecules bound to the substrate particles may also be present at a high density. As used herein, “high density” refers to a density in the range of 1,000 to 10,000,000, more preferably 10,000-1,000,000 molecules of coupling agent, ligand, MHC-peptide complex, co-stimulator molecule, or other molecule, per square micron of substrate particle surface area. In some embodiments, the density of a molecule on the substrate particle is at least about, at most about, or about 1,000, 2,000, 3,000, 4,000, 5,000, 10,000, 15,000, 20,000, 30,000, 40,000, 50,000, 100,000, 200,000, 300,000, 400,000, 500,000, or 1,000,000 molecules per square micron of substrate particle surface area, or between any two of these values. This can be measured by fluorescence staining of dissolved particles and calibrating this fluorescence to a known amount of free fluorescent molecules in solution.

In some embodiments, coupling agents can be used to associate with polymeric substrate particles and provide substrates that facilitate the modular assembly and disassembly of functional elements to the substrate particles. Coupling agents or ligands may associate with polymeric substrate particles through a variety of interactions including, but not limited to, hydrophobic interactions, electrostatic interactions and covalent coupling. In a preferred embodiment, the coupling agents are molecules that match the polymer phase hydrophile-lipophile balance. Hydrophile-lipophile balances range from 1 to 15. Molecules with a low hydrophile-lipophile balance are more lipid loving and thus tend to make a water in oil emulsion while those with a high hydrophile-lipophile balance are more hydrophilic and tend to make an oil in water emulsion. Fatty acids and lipids have a low hydrophile-lipophile balance below 10.

Any amphiphilic polymer with a hydrophile-lipophile balance in the range 1-10, more preferably between 1 and 6, most preferably between 1 and up to 5, can be used as a coupling agent. Examples of coupling agents which may associate with polymeric substrate particles via hydrophobic interactions include, but are not limited to, fatty acids, hydrophobic or amphipathic peptides or proteins, and polymers. These classes of coupling agents may also be used in any combination or ratio. In a preferred embodiment, the association of adaptor elements with nanoparticles facilitates a prolonged presentation of functional elements which can last for several weeks.

In some embodiments, coupling agents can be conjugated to affinity tags. Affinity tags are any molecular species which form highly specific, noncovalent, physiochemical interactions with defined binding partners. Affinity tags which form highly specific, noncovalent, physiochemical interactions with one another are defined herein as “complementary.” Suitable affinity tag pairs are well known in the art and include epitope/antibody, biotin/avidin, biotin/streptavidin, biotin/neutravidin, glutathione-S-transferase/glutathione, maltose binding protein/amylase, maltose binding protein/maltose, calmodulin-tag/calmodulin, His-tag/nickel or cobalt chelate, SBP-tag/streptavidin, Strep-tag/streptavidin, Strep-tag/streptactin, and TC tag/FlAsH or ReAsH biarsenical compounds. Examples of suitable epitopes which may be used for epitope/antibody binding pairs include, but are not limited to, HA, FLAG, c-Myc, glutathione-S-transferase, His6, GFP, DIG, biotin, avidin, E-tag, NE-tag, Rho1D4-tag, S-tag, C-tag, and Spot-tag. Antibodies (both monoclonal and polyclonal and antigen-binding fragments thereof) which bind to these epitopes are well known in the art. In some embodiments, one side of an affinity tag pair is bound to a substrate particle, such as by a coupling agent, and the other side of the affinity tag pair is bound to a protein or other molecule (e.g., an anti-MHC antibody or a co-stimulatory molecule) so that the binding between the two sides of the affinity tag pair causes the protein or other molecule to be bound to the substrate particle. Non-limiting examples for causing one side of an affinity tag pair to be bound to a protein are recombinant DNA technology or covalent chemical linkages.

Affinity tags that are conjugated to coupling agents allow for highly flexible, modular assembly and disassembly of functional elements which are conjugated to affinity tags which form highly specific, noncovalent, physiochemical interactions with complementary affinity tags which are conjugated to coupling agents. Adaptor elements may be conjugated with a single species of affinity tag or with any combination of affinity tag species in any ratio. The ability to vary the number of species of affinity tags and their ratios conjugated to adaptor elements allows for exquisite control over the number of functional elements which may be attached to the substrate particles and their ratios.

In another embodiment, coupling agents are coupled directly to functional elements such as antibodies, co-stimulatory molecules, inhibitor molecules, anti-MHC antibodies and other MHC-binding proteins, etc., in the absence of affinity tags, such as through direct covalent interactions. Coupling agents can be covalently coupled to at least one species of functional element. Coupling agents can be covalently coupled to a single species of functional element or with any combination of species of functional elements in any ratio. In this way, some functional elements can be bound directly to substrate particles by interaction of the coupling agent with the substrate particles.

In some embodiments, coupling agents are conjugated to at least one affinity tag that provides for assembly and disassembly of modular functional elements which are conjugated to complementary affinity tags. In some embodiments, coupling agents are fatty acids that are conjugated with at least one affinity tag. In some embodiments, the coupling agents are fatty acids conjugated with avidin or streptavidin. Avidin/streptavidin-conjugated fatty acids allow for the attachment of a wide variety of biotin-conjugated functional elements.

The coupling agents are preferably provided on, or in the surface of, microparticles or nanoparticles at a high density. As used herein, microparticles refer to particles having a largest dimension of between 0.5 and 500 μm, and nanoparticles refer to particles having a largest dimension of greater than 0.5 and less than 500 nm. This high density of coupling agents allows for coupling of the polymeric aAPCs to a variety of species of functional elements while still allowing for the functional elements to be present in high enough numbers to be efficacious.

The coupling agents may include fatty acids. Fatty acids may be of any acyl chain length and may be saturated or unsaturated. In some embodiments, the fatty acid is palmitic acid. Other suitable fatty acids include, but are not limited to, saturated fatty acids such as butyric, caproic, caprylic, capric, lauric, myristic, stearic, arachidic and behenic acid. Still other suitable fatty acids include, but are not limited to, unsaturated fatty acids such as oleic, linoleic, alpha-linolenic, arachidonic, eicosapentaenoic, docosahexaenoic and erucic acid.

The coupling agents may include hydrophobic or amphipathic peptides. Preferred peptides should be sufficiently hydrophobic to preferentially associate with the polymeric substrate particle over the aqueous environment. Amphipathic polypeptides useful as adaptor elements may be mostly hydrophobic on one end and mostly hydrophilic on the other end. Such amphipathic peptides may associate with polymeric substrate particles through the hydrophobic end of the peptide and be conjugated on the hydrophilic end to a functional group.

Coupling agents may include hydrophobic polymers. Examples of hydrophobic polymers include, but are not limited to, polyanhydrides, poly(ortho)esters, and polyesters such as polycaprolactone.

C. Functional Molecules Bound to Substrate Particles

Embodiments disclosed herein have functional elements, also referred to as functional molecules, bound to substrate particles. Functional elements may include, for example, MHC-binding proteins, anti-MHC antibodies, T cell co-stimulatory molecules, inhibitory molecules, T cell adhesion molecules, chemoattractants, T cell receptor ligands, and other molecules described herein that affect the function of the aAPC, such as by targeting aAPCs to T cells and to mimic interactions that occur between natural APCs and T cells to elicit efficient activation and expansion of T cells.

Substrate particles may be associated with a single species of functional element or may be associated with any combination of disclosed functional elements in any ratio. In some embodiments, functional elements are associated with substrate particles through coupling agents which directly associate with the substrate particles. Functional elements may be directly or covalently coupled to coupling agents or may bind to coupling agents through complementary affinity tags conjugated to the coupling agents and functional elements. Multiple different species of functional elements may be associated with substrate particles, for instance, by conjugating each species of functional element to a separate species of affinity tag. These functional elements may then associate with substrate particles coated with coupling agents conjugated to an appropriate ratio of complementary affinity tags. Multiple species of functional elements may also be associated with substrate particles by covalently coupling each species of functional element at a desired ratio to coupling agents. In some embodiments, functional elements are conjugated to biotin. Biotin conjugation allows the functional elements to interact with coupling agents conjugated with avidin, neutravidin or streptavidin.

Functional elements bound to substrate particles can include in some embodiments antigen-specific T cell receptor activators. Antigen molecules are recognized by the immune system after internal processing by natural APCs (Lanzavecchia, Curr. Opin. Immunol., 8:348-54 (1996)). In order to present an antigen, the antigen is broken down into small peptidic fragments by enzymes contained in vesicles in the cytoplasm of the APCs (Wick, et al., Immunol. Rev., 172:153-62 (1999); Lehner, et al., Curr. Biol., 8: R605-8 (1998); Braciale, Curr. Opin. Immunol., 4:59-62 (1992)). The enzymes are part of a complex of proteolytic enzymes called a proteasome. Most cells have several different types of proteasomes with differing combinations of specificities, which they use to recycle their intracellular proteins. The peptides produced by the proteasomes are generated in the cytosol and transported into the Golgi, where they are linked to cellular major histocompatibility complex (MHC) molecules. These are referred to as human leukocyte antigens, or “HLAs”, in human. MHC and HLA are used interchangeably herein unless specified otherwise.

In some embodiments, the substrate particles described herein are bound to endogenous antigen-presenting molecules having determinants which match that of a selected subject or which match any known antigen-presenting molecule determinants. The antigen-presenting molecules may be MHC/HLA class I or class II molecules. There are two types of HLA molecules used for antigen presentation, class I and class II molecules. HLA class I molecules are expressed on the surface of all cells and HLA class II are expressed on the surface of a specialized class of cells called professional APCs. HLA class II molecules bind primarily to peptides derived from proteins made outside of an APC, but can present self (endogenous) antigens. In contrast, HLA class I molecules bind to peptides derived from proteins made inside a cell, including proteins expressed by an infectious agent (e.g., such as a virus) in the cell and by a tumor cell. When the HLA class I proteins reach the surface of the cell these molecules will thus display any one of many peptides derived from the cytosolic proteins of that cell, along with normal “self” peptides being synthesized by the cell. Peptides presented in this way are recognized by T-cell receptors which engage T-lymphocytes in an immune response against the antigens to induce antigen-specific cellular immunity. Embodiments disclosed herein include substrate particles bound, directly or indirectly, to endogenous HLA class I or II molecules displaying peptides of antigens produced in the cell.

Class I transplantation antigens of the major histocompatibility complex (MHC) or HLA are cell surface glycoproteins which present antigens to cytotoxic T-cells. They are heterodimeric and composed of a polymorphic, MHC-encoded, approximately 45 kD heavy chain, which is non-covalently associated with an approximately 12 kD β-2 microglobulin (β-2m) light chain.

The extracellular portion of the MHC Class I heavy chain is divided into three domains, α-1, α-2, and α-3, each approximately 90 amino acids long and encoded on separate exons. The α-3 domain and β-2m are relatively conserved and show amino-acid sequence homology to immunoglobulin constant domains. The polymorphic α-1 and α-2 domains show no significant sequence homology to immunoglobulin constant or variable region, but do have weak sequence homology to each other. The membrane-distal polymorphic α-1 (approximately 90 amino acids) and α-2 (approximately 92 amino acids) domains each include four anti-parallel, β-pleated sheets bordered by one α-helical regions, (the first from the α-1 and the second from the α-2 domain). The α-2 domain is attached to the less-polymorphic, membrane-proximal α-3 (approximately 92 amino acids) domain which is followed by a conserved transmembrane (25 amino acids) and an intra-cytoplasmic (approximately 30 amino acids) segment. The rat, mouse, and human Class I MHC molecules are believed to have similar structural characteristics based upon known nucleotide sequences of the various MHC Class I molecules.

The classical class I gene family includes the highly polymorphic human class I molecules HLA-A, -B, and -C, and murine class I (i.e., H-2) molecules D, K, and L. A series of structural relatives (non-classical class I molecules) has been found in humans (e.g., HLA-E, -F, -G, -H, -I, and -J; and CD1) and mice (Q, T, M, and CD1) (Shawar, et al., Annu. Rev. Immunol., 12:839-880 (1994)). These molecules have the typical structure of an antigen-presenting molecule, where a polymorphic heavy chain is noncovalently associated with the conserved β2-M subunit.

Substrate particles may be bound to MHC molecules, MHC-peptide complexes (including MHC I-peptide and MHC II-peptide complexes), T cell co-stimulatory molecules, T cell inhibitory molecules, and other functional molecules, including but not limited to adhesion molecules, modulation molecules, and inhibitory molecules. Accessory molecules may be bound to substrate particles for the purpose of stabilizing an interaction between a T-cell receptor and an MHC or MHC-peptide complex. Suitable accessory molecules may include, but are not limited to, LFA-1, CD49d/29(VLA-4), CD 11a/18, CD54(ICAM-1), and CD106(VCAM) and antibodies to their ligands.

Co-stimulatory molecules may bound to substrate particles for the purpose of stimulating or activating a TCR. Suitable co-stimulatory molecules may include, but are not limited to, B7-1, B7-2, CD5, CD9, CD40, CD70, CD80, CD86, ICOS-L, OX40-L, IL-2, IL-7, 4-1BBL, IFN-gamma, IL-12, IL-15, IL-17, IL-18, IL-22, TNF-α, LFA-3, ICAM-1, ICOS-L, GITR-L, anti-CD28 agonistic antibody, anti-CTLA-4 antagonistic antibody, anti-ICOS agonistic antibody, anti-PDL1-antagonistic antibody, anti-PDL-2 antagonistic antibody, anti-B7-H3-receptor antagonistic antibody, and anti-B7-H4 receptor antagonistic antibody. Co-stimulatory molecules may also include, but are not limited to, polypeptides, including antibodies, that specifically bind and activate one or more of the following T cell membrane surface molecules: CD28, OX40, 4-1BB, CD27, ICOS, and GITR. Any of these co-stimulatory molecules can be used in combinations of 2, 3, 4, 5, 6, 7, 8, 9, or 10 or more co-stimulatory molecules.

Inhibitory molecules may be bound to substrate particles for the purpose of down regulating a T-cell response via interaction with a TCR. Suitable inhibitory molecules include, but are not limited to B7.1wa, CTLA-4 binding proteins, PD-L1, HVEM, PDL-2, B7-H3, B7-H4, OX-2, TGF-beta1, IL-10, IL-4, natural and recombinant anti-CTLA-4 agonistic antibody, anti-PD-1 agonistic antibody, Anti-B7-H3 receptor agonistic antibody, Anti-B7-H4 receptor antibody, anti-CD28 antagonistic antibody, and anti-ICOS antagonistic antibody. Any of these inhibitory molecules may be used in combinations of 2, 3, 4, 5, 6, 7, 8, 9, or 10 or more inhibitory molecules.

In some embodiments for use in methods of inducing immune tolerance, MHC-peptide complexes bound to substrate particles may include, but are not limited to, peptides from self antigens (e.g. antigenic peptides, of insulin, insulin, GAD, GAD65, HSP, thyroglobulin, nuclear proteins, acetylcholine receptor, collagen, TSHR, ICA512(IA-2) and IA-2β (phogrin), carboxypeptidase H, ICA69, ICA12, thyroid peroxidase), histocompatibility allo- and xeno-antigens, and allergenic proteins. Furthermore, such antigens may be selected from the group consisting of a peptide derived from the recipient for graft versus host diseases, a cancer cell-derived peptide, a donor derived peptide, a pathogen-derived molecule, a peptide derived by epitope mapping, a self-derived molecule, a self-derived molecule that has sequence identity with the pathogen-derived antigen, the sequence identity having a range selected from the group consisting of between 5 and 100%, 15 and 100%, 35 and 100%, and 50 and 100%.

In some embodiments, aAPCs may be complexed to a solid support in addition to the T cell. This provides a means to anchor the aAPC so that it and any T cell binding to it can be preferentially captured and isolated from extraneous matter. In such case, the solid support may be a glass or magnetic bead that is coated with, for example, a lipid mono layer that is bound to the bead by, for example, a linker. The solid support may additionally have noncovalently bound accessory molecules associated with the lipid monolayer such as binding molecules that recognize and bind to molecules, such as labels or tags, associated with the aAPC. In another embodiment, the binding molecules may be covalently bound to the solid support by a linker.

The aAPCs disclosed herein may have a size of between about 0.2 μm and 50 μm for administration with a needle. For needle-injected aAPCs, the aAPCs may have any appropriate dimensions so long as the larges dimension of the aAPC permits the microsphere to move through a needle. The aAPCs that are used to contact T cells in vitro may be larger, including up to 500 μm in size.

In some embodiments, aAPCs disclosed herein are at least about, at most about, or about 0.1, 0.2, 0.3, 0.4, 0.5, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 40, 50, 60, 70, 80, 90, 100, 150, 200, 250, 300, 350, 400, 450, or 500 μm in size (i.e., in their largest dimension), or between any two of these values. In a preferred embodiment, the aAPC is between about 2 μm and 20 μm in diameter. In some embodiments, the aAPCs disclosed herein are at least about, at most about, or about 1, 5, 10, 20, 30, 40, 50, 60, 70, 80, 90, 100, 125, 150, 175, 200, 225, 250, 275, 300, 325, 350, 375, 400, 425, 450, 475, or 500 nm in size, or between any two of these values.

Adhesion molecules may be used as functional elements bound to substrate particles for the purpose of enhancing the binding association between the aAPCs and T cells. Suitable adhesion molecules include, but are not limited to, LFA-1, CD49d/29(VLA-4), CD11a/18, CD54(ICAM-1), and CD106(VCAM) and antibodies to their ligands. Other suitable adhesion molecules include antibodies to selectins L, E, and P. Any of these adhesion molecules can be used in combinations of 2, 3, 4, 5, 6, 7, 8, 9, or 10 or more adhesion molecules.

D. MHC Binding Polypeptides

Embodiments disclosed herein include aAPCs that have MHC-peptide complexes bound to substrate particles. In some embodiments, the MHC-peptide complexes are bound to the substrate particles via a polypeptide that is capable of specifically binding MHC. Such polypeptides include in some embodiments anti-MHC antibodies and functional fragments thereof. The anti-MHC antibodies may specifically bind human MHC class I, human MHC class II, or non-human animal equivalents of these MHCs. Many anti-MHC antibodies are known and commercially available in the art. Suitable anti-MHC antibodies include, but are not limited to anti-MHC class I antibodies available from Novus Biologicals and designated OX18, 2G5, ERMP42, ER-HR52, JF10-38, h58A, DG-H58A, MEM-E/06, F21-2, MEM-E/02, MEM-E/08, and A4. Suitable anti-MHC antibodies also include anti-MHC class II antibodies, available from Novus Biologicals and designated M5/115.15.2, CVW20, ER-TR2, M5/114, H42A, TH14B, CA2.1c12, TH81A, AP-MAB0874, NIMR-4, HAL16A, and P-TH81A5. Suitable anti-MHC antibodies include, but are not limited to anti-MHC antibodies available from abcam and designated MRC OX-6, NIMR-4, ab23990, ab25333, ab55152, ab134189, ab180779, ab25228, ab15680, ab15681, ab25681, M5/114, M5/114.15.2, ab116378. Suitable anti-MHC antibodies may also include HC-10 and w6/32. Many other anti-MHC antibodies are readily available and may be used or adapted for use in methods described herein. Particularly suitable antibodies are those that bind epitopes located a suitable distance away from the peptide-binding site so as to avoid interference with peptide binding by the MHC complex. Anti-MHC antibodies to be used in embodiments disclosed herein may be monoclonal or polyclonal. Anti-MHC antibodies to be used in embodiments disclosed herein may specifically bind any of the polypeptides that are included in MHC complexes, including class I β₂ microglobulin, class I α chain, class II α chain, or class II β chain. Anti-MHC antibodies to be used in embodiments disclosed herein may be, for example, human antibodies, rabbit, mouse, goat, or other antibodies, and may be humanized or chimeric. Mixtures of anti-MHC antibodies and MHC binding proteins may also be used to provide for efficient pull-down of endogenous MHC-peptide complexes from cell lysates and other preparations. Variants of available antibodies, such as humanized, chimeric, scFv, and other engineered antibody constructs can also be used. Polypeptides capable of binding MHC may be engineered or modified to avoid eliciting an immune response against the MHC-binding polypeptide itself.

In some embodiments, the polypeptide capable of binding MHC is a viral protein. For example, adenoviral protein E3-19k binds MHC class I protein and can be used to pull down MHC I-peptide complexes from a lysate or other preparation. Other suitable MHC binding proteins include, but are not limited to, US2, US3, US 11, and other proteins disclosed in Yewdell & Bennink, Annu. Rev. Cell Dev. Biol., 15:579-606 (1999), which is incorporated herein by reference.

E. Antigens

Embodiments disclosed herein include endogenous MHC molecules bound to peptides. The MHC-peptide complexes can be bound to the surface of substrate particles to make aAPCs effective for activating and expanding T cell populations that specifically target antigens from which the peptides are derived. Target antigens may include, for example, cancer antigens, viral antigens, bacterial antigens. and/or self antigens. In some embodiments, one or more cancer cells expressing a target antigen can be used as a source of endogenous MHC I-peptide complexes to be bound to substrate particles, which can then be used as aAPCs to activate and proliferate cytotoxic T cells specific for the target antigen. Similar approaches can be used to target cells infected with a virus-viral-infected cells can be used as a source for MHC I-peptide complexes that include peptides from viral antigens.

Tumor antigens that may be targeted using embodiments disclosed herein include, but are not limited to, tumor-specific antigens or tumor-associated antigens, including alpha-actinin-4, Bcr-Abl fusion protein, Casp-8, beta-catenin, cdc27, cdk4, cdkn2a, coa-1, dek-can fusion protein, EF2, ETV6-AML1 fusion protein, LDLR-fucosyltransferaseAS fusion protein, HLA-A2, HLA-A11, hsp70-2, KIAAO205, Mart2, Mum-1, 2, and 3, neo-PAP, myosin class I, OS-9, pml-RARα fusion protein, PTPRK, K-ras, N-ras, Triosephosphate isomeras, Bage-1, Gage 3, 4, 5, 6, 7, GnTV, Herv-K-mel, Lage-1, Mage-A1, 2, 3, 4, 6, 10, 12, Mage-C2, NA-88, NY-Eso-1/Lage-2, SP17, SSX-2, and TRP2-Int2, MelanA (MART-I), gp100 (Pmel 17), tyrosinase, TRP-1, TRP-2, MAGE-1, MAGE-3, BAGE, GAGE-I, GAGE-2, p15(58), CEA, RAGE, NY-ESO (LAGE), SCP-1, Hom/Mel-40, PRAME, p53, H-Ras, HER-2/neu, BCR-ABL, E2A-PRL, H4-RET, IGH-IGK, MYL-RAR, Epstein Barr virus antigens, EBNA, human papillomavirus (HPV) antigens E6 and E7, TSP-180, MAGE-4, MAGE-5, MAGE-6, p185erbB2, p180erbB-3, c-met, nm-23H1, PSA, TAG-72-4, CA 19-9, CA 72-4, CAM 17.1, NuMa, K-ras, β-Catenin, CDK4, Mum-1, p16, TAGE, PSMA, PSCA, CT7, telomerase, 43-9F, 5T4, 791Tgp72, α-fetoprotein, 13HCG, BCA225, BTAA, CA 125, CA 15-3 (CA 27.29\BCAA), CA 195, CA 242, CA-50, CAM43, CD68\KP1, CO-029, FGF-5, G250, Ga733 (EpCAM), HTgp-175, M344, MA-50, MG7-Ag, MOV18, NB\170K, NY-CO-1, RCAS1, SDCCAG16, TA-90 (Mac-2 binding protein\cyclophilin C-associated protein), TAAL6, TAG72, TLP, and TPS.

Viral antigens that may be targeted using embodiments disclosed herein include, without limitation, antigens from any of the following viral families: Arenaviridae, Arterivirus, Astroviridae, Baculoviridae, Badnavirus, Barnaviridae, Birnaviridae, Bromoviridae, Bunyaviridae, Caliciviridae, Capillovirus, Carlavirus, Caulimovirus, Circoviridae, Closterovirus, Comoviridae, Coronaviridae (e.g., Coronavirus, such as severe acute respiratory syndrome (SARS) virus), Corticoviridae, Cystoviridae, Deltavirus, Dianthovirus, Enamovirus, Filoviridae (e.g., Marburg virus and Ebola virus (e.g., Zaire, Reston, Ivory Coast, or Sudan strain)), Flaviviridae, (e.g., Hepatitis C virus, Dengue virus 1, Dengue virus 2, Dengue virus 3, and Dengue virus 4), Hepadnaviridae, Herpesviridae (e.g., Human herpesvirus 1, 3, 4, 5, and 6, and Cytomegalovirus), Hypoviridae, Iridoviridae, Leviviridae, Lipothrixviridae, Microviridae, Orthomyxoviridae (e.g., Influenzavirus A and B and C), Papovaviridae, Paramyxoviridae (e.g., measles, mumps, and human respiratory syncytial virus), Parvoviridae, Picornaviridae (e.g., poliovirus, rhinovirus, hepatovirus, and aphthovirus), Poxyiridae (e.g., vaccinia and smallpox virus), Reoviridae (e.g., rotavirus), Retroviridae (e.g., lentivirus, such as human immunodeficiency virus (HIV) I and HIV 2), Rhabdoviridae (for example, rabies virus, measles virus, respiratory syncytial virus, etc.), Togaviridae (for example, rubella virus, dengue virus, etc.), and Totiviridae. Suitable viral antigens also include all or part of Dengue protein M, Dengue protein E, Dengue D1NS1, Dengue D1NS2, and Dengue D1NS3.

Viral antigens may be from a particular strain such as a papilloma virus, a herpes virus, i.e. herpes simplex 1 and 2; a hepatitis virus, for example, hepatitis A virus (HAV), hepatitis B virus (HBV), hepatitis C virus (HCV), the delta hepatitis D virus (HDV), hepatitis E virus (HEV) and hepatitis G virus (HGV), the tick-borne encephalitis viruses; parainfluenza, varicella-zoster, cytomeglavirus, Epstein-Barr, rotavirus, rhinovirus, adenovirus, coxsackieviruses, equine encephalitis, Japanese encephalitis, yellow fever, Rift Valley fever, and lymphocytic choriomeningitis.

Bacterial antigens that may be targeted using embodiments disclosed herein can include without limitation those from any bacteria including, but not limited to, Actinomyces, Anabaena, Bacillus, Bacteroides, Bdellovibrio, Bordetella, Borrelia, Campylobacter, Caulobacter, Chlamydia, Chlorobium, Chromatium, Clostridium, Corynebacterium, Cytophaga, Deinococcus, Escherichia, Francisella, Halobacterium, Heliobacter, Haemophilus, Hemophilus influenza type B (HIB), Hyphomicrobium, Legionella, Leptspirosis, Listeria, Meningococcus A, B and C, Methanobacterium, Micrococcus, Myobacterium, Mycoplasma, Myxococcus, Neisseria, Nitrobacter, Oscillatoria, Prochloron, Proteus, Pseudomonas, Phodospirillum, Rickettsia, Salmonella, Shigella, Spirillum, Spirochaeta, Staphylococcus, Streptococcus, Streptomyces, Sulfolobus, Thermoplasma, Thiobacillus, and Treponema, Vibrio, and Yersinia.

Parasite antigens that may be targeted using embodiments disclosed herein include without limitation those from Cryptococcus neoformans, Histoplasma capsulatum, Candida albicans, Candida tropicalis, Nocardia asteroides, Rickettsia ricketsii, Rickettsia typhi, Mycoplasma pneumoniae, Chlamydial psittaci, Chlamydial trachomatis, Plasmodium falciparum, Trypanosoma brucei, Entamoeba histolytica, Toxoplasma gondii, Trichomonas vaginalis and Schistosoma mansoni. These include Sporozoan antigens, Plasmodian antigens, such as all or part of a Cireumsporozoite protein, a Sporozoite surface protein, a liver stage antigen, an apical membrane associated protein, or a Merozoite surface protein.

Antigens targeted in embodiments for immune tolerance disclosed herein may also be an allergen or environmental antigen, such as, but not limited to, an antigen derived from naturally occurring allergens such as pollen allergens (tree-, herb, weed-, and grass pollen allergens), insect allergens (inhalant, saliva and venom allergens), animal hair and dandruff allergens, and food allergens. Important pollen allergens from trees, grasses and herbs originate from the taxonomic orders of Fagales, Oleales, Pinales and platanaceae including La. birch (Betula), alder (Alnus), hazel (Corylus), hornbeam (Carpinus) and olive (Olea), cedar (Cryptomeria and Juniperus), Plane tree (Platanus), the order of Poales including i.e. grasses of the genera Lolium, Phleum, Poa, Cynodon, Dactylis, Holcus, Phalaris, Secale, and Sorghum, the orders of Asterales and Urticales including i.a. herbs of the genera Ambrosia, Artemisia, and Parietaria. Other allergen antigens that may be used include allergens from house dust mites of the genus Dermatophagoides and Euroglyphus, storage mite e.g Lepidoglyphys, Glycyphagus and Tyrophagus, those from cockroaches, midges and fleas e.g. Blatella, Periplaneta, Chironomus and Ctenocepphalides, those from mammals such as cat, dog and horse, birds, venom allergens including such originating from stinging or biting insects such as those from the taxonomic order of Hymenoptera including bees (superfamily Apidae), wasps (superfamily Vespidea), and ants (superfamily Formicoidae). Still other allergen antigens that may be used include inhalation allergens from fungi such as from the genera Alternaria and Cladosporium.

Antigens targeted in embodiments for immune tolerance disclosed herein may also be a self-antigen or an autoantigen. Antigens may be antigens of any autoimmune or inflammatory disease or disorder including, but not limited to, diabetes mellitus, arthritis (including rheumatoid arthritis, juvenile rheumatoid arthritis, osteoarthritis, psoriatic arthritis), multiple sclerosis, myasthenia gravis, systemic lupus erythematosis, autoimmune thyroiditis, dermatitis (including atopic dermatitis and eczematous dermatitis), psoriasis, Sjogren's Syndrome, including keratoconjunctivitis sicca secondary to Sjogren's Syndrome, alopecia greata, allergic responses due to arthropod bite reactions, Crohn's disease, ulcer, iritis, conjunctivitis, keratoconjunctivitis, ulcerative colitis, asthma, allergic asthma, cutaneous lupus erythematosus, scleroderma, vaginitis, proctitis, drug eruptions, leprosy reversal reactions, erythema nodosum leprosum, autoimmune uveitis, allergic encephalomyelitis, acute necrotizing hemorrhagic encephalopathy, idiopathic bilateral progressive sensorineural hearing loss, aplastic anemia, pure red cell anemia, idiopathic thrombocytopenia, polychondritis, Wegener's granulomatosis, chronic active hepatitis, Stevens-Johnson syndrome, idiopathic spree, lichen planus, Crohn's disease, Graves opthalmopathy, sarcoidosis, primary biliary cirrhosis, uveitis posterior, and interstitial lung fibrosis.

Autoantigens of the present invention include, but are not limited to, at least a portion of a thyroid-stimulating hormone receptor, pancreatic P cell antigens, epidermal cadherin, acetyl choline receptor, platelet antigens, nucleic acids, nucleic acid protein complexes, myelin protein, thyroid antigens, joint antigens, antigens of the nervous system, salivary gland proteins, skin antigens, kidney antigens, heart antigens, lung antigens, eye antigens, erythrocyte antigens, liver antigens and stomach antigens. Examples of antigens involved in autoimmune disease include glutamic acid decarboxylase 65 (GAD 65), native DNA, myelin basic protein, myelin proteolipid protein, acetylcholine receptor components, thyroglobulin, and the thyroid stimulating hormone (TSH) receptor. Examples of antigens involved in graft rejection include antigenic components of the graft to be transplanted into the graft recipient such as heart, lung, liver, pancreas, kidney, and neural graft components.

F. Cytokines and Growth Factors

In some embodiments, substrate particles include additional signaling molecules such as, for example, cytokines and growth factors, which may be used contribute to activation and proliferation of T cells.

Cytokines, the largest class of immunoregulatory molecules, are secreted by activated APCs after T cell encounters and impact expansion, survival, effector function, and memory of stimulated T cells. Typically, cytokines are added to cultures exogenously and administered systemically to patients following re-infusion of T cells, however, such systemic administration can be associated with acute toxicity as in the case of IL-2 in clinical trials (Fyfe, et al., J. Clin. Oncol., 13(3):688-96 (1995)). While exogenous addition of cytokines is a simple strategy to augment signaling, it has been discovered that paracrine release of cytokines from polymeric aAPCs represents a more efficacious strategy.

In some embodiments, the aAPCs disclosed herein comprise substrate particles that contain cytokines encapsulated in or incorporated into substrate particles, including polymeric substrate particles. Suitable cytokines include, but are not limited to, hematopoietic growth factors, interleukins, interferons, immunoglobulin superfamily molecules, tumor necrosis factor family molecules and chemokines. Preferred cytokines include, but are not limited to, granulocyte macrophage colony stimulating factor (GM-CSF), tumor necrosis factor alpha (TNFα), tumor necrosis factor beta (TNFβ), macrophage colony stimulating factor (M-CSF), interleukin-1 (IL-1), interleukin-2 (IL-2), interleukin-4 (IL-4), interleukin-5 (IL-5), interleukin-6 (IL-6), interleukin-10 (IL-10), interleukin-12 (IL-12), interleukin-15 (IL-15), interleukin-21 (IL-21), interferon alpha (IFNα), interferon beta (IFNβ), interferon gamma (IFNγ), and IGIF, and variants and fragments thereof.

Suitable chemokines include, but are not limited to, an alpha-chemokine or a beta-chemokine, including, but not limited to, a C5a, interleukin-8 (IL-8), monocyte chemotactic protein 1alpha (MIP1α), monocyte chemotactic protein 1 beta (MIP1β), monocyte chemoattractant protein 1 (MCP-1), monocyte chemoattractant protein 3 (MCP-3), platelet activating factor (PAFR), N-formyl-methionyl-leucyl-[3H]phenylalanine (FMLPR), leukotriene B4, gastrin releasing peptide (GRP), RANTES, eotaxin, lymphotactin, IP10, I-309, ENA78, GCP-2, NAP-2 and MGSA/gro, and variants and fragments thereof.

Cytokines that are encapsulated in or incorporated into the polymeric microparticles may be first stabilized by complexing or mixing with preservation agents. Suitable preservation agents include, but are not limited to, trehalose, mannitol, PEG 400, PEG 2000, PEG 3350, albumins, phosphatidyl-choline, gelatin, tweens and pluronics.

In some embodiments, cytokines and chemokines can be used in any combination of 2, 3, 4, 5, 6, 7, 8, 9, 10, 11, 12, 13, 14, 15 or more different cytokines and/or chemokines.

### III. T Cells and Immunotherapy

Immunotherapeutic treatment aims to use the body's own immune defense mechanisms to target specific abnormal cells while minimizing nonspecific toxicity. Immunotherapy can be used to prime and amplify antigen-specific lymphocytes either in vivo (active immunotherapy) or ex vivo prior to their infusion (adoptive immunotherapy). Adoptive immunotherapy is a procedure whereby an individual's own lymphocytes are expanded ex vivo and re-infused back into the body. Both adoptive and active immunotherapy can be used as therapeutic strategies for the treatment of viral infection (Papadopoulos, et al., N. Engl. J. Med, 330(17):1185-91 (1994); Savoldo, et al., Leuk Lymphoma, 39(5-6):455-64 (2000)), autoimmune disease (Hori, et al., Adv. Immunol., 81:331-71 (2003); Karim, et al., J. Immunol., 172(2):923-8 (2004)), or cancer (Dudley, et al., Nat. Rev. Cancer, 3(9):666-75 (2003); Riddell, et al., Cancer Control, 9(2):114-22 (2002); Yee, et al., Proc. Natl. Acad. Sci. USA., 99(25):16168-73 (2002)).

The process of antigen-specific activation, expansion and differentiation that is essential to the establishment of immunity is determined to a large extent by the interaction between T cells and antigen-presenting cells (APCs). Efficient stimulation of antigen-specific T cells depends on the interaction of the T cell antigen receptor (TCR) with specific antigen in the form of a peptide/major histocompatibility complex (pMHC) on APCs. In addition to this recognition signal, co-stimulation through the B7 family of receptors on APCs, which engage the CD28 receptor and related receptors on T cells, is known to amplify antigen-specific T cell responses (Michel, et al., Immunity, 15(6):935-45 (2001)).

T cell activation and function is also influenced by cytokines, the largest class of immunoregulatory molecules. Cytokines are secreted by activated antigen presenting cells after T cell encounters and impact expansion, survival, effector function, and memory of stimulated T cells (Pardoll, Nat. Rev. Immunol., 2(4):227-38 (2002); Fyfe, et al., J. Clin. Oncol., 13(3):688-96 (1995); Schluns, et al., Nat. Rev. Immunol, 3(4):269-79 (2003)).

A. Active Immunotherapy

In embodiments disclosed herein, aAPCs disclosed herein are used for active immunotherapy. For active immunotherapy, the aAPCs are administered directly to the subject to be treated in the same manner as a vaccine. Thus, in such methods, the contacting of T cells with aAPCs occurs in vivo in a subject's body. In general, methods of administering polymeric substrate particles and vaccines are well known in the art. Any acceptable method known to one of ordinary skill in the art may be used to administer a formulation to the subject. The administration may be localized (i.e., to a particular region, physiological system, tissue, organ, or cell type) or systemic. aAPCs can be administered by a number of routes including, but not limited to, injection: intravenous, intraperitoneal, intramuscular, or subcutaneous, to a mucosal surface (oral, sublingual or buccal, nasal, rectal, vaginal, pulmonary), or transdermal. In some embodiments, the injections can be given at multiple locations. The aAPCs can also be administered directly to an appropriate lymphoid tissue, such as the spleen, lymph nodes or mucosal-associated lymphoid tissue.

Administration of the formulations may be accomplished by any acceptable method which allows an effective amount of the aAPCs to reach their target. The particular mode selected will depend upon factors such as the particular formulation, the severity of the state of the subject being treated, and the dosage required to induce an effective immune response. As generally used herein, an “effective amount” is that amount which is able to induce an immune response in the treated subject. The actual effective amounts of aAPCs can vary according to factors including the specific antigen or combination thereof being utilized, the density and/or nature of the associated co-stimulatory molecules, the release characteristics of the encapsulated cytokines, the particular composition formulated, the mode of administration, and the age, weight, condition of the subject being treated, as well as the route of administration and the disease or disorder.

B. Adoptive Immunotherapy

In some embodiments, a source of T cells for adoptive immunotherapy can be obtained from a subject to be treated for use in adoptive immunotherapy in an organism in which an immune response can be elicited, e.g., mammals. Examples of subjects include humans, dogs, cats, mice, rats, and transgenic species thereof, although humans are preferred. T cells can be obtained from a number of sources, including peripheral blood leukocytes, bone marrow, lymph node tissue, spleen tissue, and tumors. In a preferred embodiment, peripheral blood leukocytes are obtained from an individual by leukopheresis. To isolate T cells from peripheral blood leukocytes, it may be necessary to lyse the red blood cells and separate peripheral blood leukocytes from monocytes by, for example, centrifugation through, e.g., a PERCOLL™ gradient.

A specific subpopulation of T cells, such as CD4+ or CD8+ T cells, can be further isolated by positive or negative selection techniques. For example, negative selection of a T cell population can be accomplished with a combination of antibodies directed to surface markers unique to the cells negatively selected. One suitable technique includes cell sorting via negative magnetic immunoadherence, which utilizes a cocktail of monoclonal antibodies directed to cell surface markers present on the cells negatively selected. For example, to isolate CD4+ cells, a monoclonal antibody cocktail typically includes antibodies to CD14, CD20, CD11b, CD16, HLA-DR, and CD8. The process of negative selection results in an essentially homogenous population of the desired T cell population.

AAPCs are customized according to the subject and the condition or disease to be treated. In one embodiment, the aAPCs contain at least one polyclonal T cell receptor activator, such as an anti-T cell receptor antibody. Polyclonal T cell activation can be useful because it can expand a T cell population more quickly than antigen-specific methods. The expanded polyclonal T cells can then be sorted to select for T cells with a specificity for the epitopes of interest. In another embodiment, the aAPCs contain MHC class I or MHC class II molecules bound to antigens of interest for antigen-specific T cell activation. The MHC polypeptides used in the aAPCs are preferably selected to match the MHC alleles expressed by the subject to be treated. As MHC polypeptides used in methods disclosed herein include endogenous MHC polypeptides from a patient's own cells, embodiments disclosed herein do not require taking steps to ensure that matching MHC alleles are used. The antigen is selected based on the condition or disease to be treated or prevented. The antigen may be derived from the subject to be treated. For example, cells from which the MHC-peptide complexes are obtained for creation of aAPCs in embodiments disclosed herein may be obtained from the patient itself, the antigens are those that are produced by the cell, and peptides from those antigens are bound to MHC molecules through the cell's endogenous processes.

For adoptive immunotherapy embodiments, the selected T cells are then contacted ex vivo in appropriate medium with the aAPCs. AAPCs are used in amounts effective to cause activation and proliferation of T cells. The T cells are contacted with the aAPCs for periods of time necessary for expansion of the T cells. It may be advantageous to maintain long-term culture of a population of T cells following the initial activation and stimulation, by separating the T cells from the stimulus after a period of about 12 to about 14 days. In certain embodiments, it may be desirable to separate the T cells from the stimulus after a period of about 2, 3, 4, 5, 6, 7, 8, 9, 10, or 11 days, or any range derivable therein. In certain embodiments, it may be desirable to separate the T cells from the stimulus after a period of less than one day, such as after about an hour, or 2, 3, 4, 5, 6, 7, 8, 9, 10, 11, 12, 13, 14, 15, 16, 17, 18, 19, 20, 21, 22, or 23 hours, or any range derivable therein. The rate of T cell proliferation is monitored periodically (e.g., daily) by, for example, examining the size or measuring the volume of the T cells, such as with a Coulter Counter. In this regard, a resting T cell has a mean diameter of about 6.8 microns, and upon initial activation and stimulation, in the presence of the stimulating ligand, the T cell mean diameter will increase to over 12 microns by day 4 and begin to decrease by about day 6. The T cells may be stimulated through multiple rounds of activation by the aAPCs. For example, when the mean T cell diameter decreases to approximately 8 microns, the T cells may be reactivated and re-stimulated to induce further proliferation of the T cells. Alternatively, the rate of T cell proliferation and time for T cell re-stimulation can be monitored by assaying for the presence of cell surface molecules, such as, CD154, CD54, CD25, CD137, CD134, which are induced on activated T cells.

Following activation and expansion of the T cells, they are administered to the subject in amounts effective to induce an immune response. The T cells may be administered separately from, or in combination with, the aAPCs. The immune response induced in the animal by administering the compositions may include cellular immune responses mediated by CD8+ T cells, capable of killing tumor and infected cells, and CD4+ T cell responses. Humoral immune responses, mediated primarily by B cells that produce antibodies following activation by CD4+ T cells, may also be induced. In a preferred embodiment, the immune response is mediated by cytolytic CD8+ T cells. A variety of techniques which are well known in the art may be used for analyzing the type of immune responses induced by the compositions and methods disclosed herein (Coligan et al., Current Protocols in Immunology, John Wiley & Sons Inc. (1994)).

C. Adoptive Immunotherapy for Immune Tolerance

Adoptive immunotherapy may also be used to treat or prevent conditions associated with undesirable activation, over-activation or inappropriate or aberrant activation of an immune response, as occurs in conditions including autoimmune disorders and diseases, allergic reactions, graft rejection and graft-versus-host disease. In one embodiment, undesirable or aberrant antigen-specific immune responses are treated or prevented by adoptive immunotherapy using “regulatory” T cells (Tregs) activated by the compositions and methods disclosed herein.

Immunological self-tolerance is critical for the prevention of autoimmunity and maintenance of immune homeostasis. The ability of the immune system to discriminate between self and non-self is controlled by mechanisms of central and peripheral tolerance. Central tolerance involves deletion of self-reactive T lymphocytes in the thymus at an early stage of development (Rocha, et al., Science, 251:1225-1228 (1991); Kisielow, et al., Nature, 333:742-746 (1988)). Several mechanisms of peripheral tolerance have been described, including T cell anergy and ignorance (Schwartz, Science, 248:1349-1356 (1990); Miller, et al., Immunol. Rev., 133:131-150 (1993)). Studies have provided firm evidence for the existence of a unique CD4+CD25+ population of professional regulatory/suppressor T cells that actively and dominantly prevent both the activation as well as the effector function of autoreactive T cells that have escaped other mechanisms of tolerance (Sakaguchi, et al., J. Immunol., 155:1151-1164 (1995); Takahashi, et al., Int. Immunol., 10:1969-1980 (1998); Itoh, et al., J. Immunol., 162:5317-5326 (1999)). The elimination or inactivation of these cells resulted in severe autoimmune disease, and was also found to enhance immune responses to alloantigens and even tumors (Sakaguchi, et al., J. Immunol., 155:1151-1164 (1995); Itoh, et al., J. Immunol., 162:5317-5326 (1999); Shimizu, et al., J. Immunol., 163:5211-5218 (1999)). Autoantigen-specific regulatory T (Treg) cells actively regulate autoimmunity and induce long term tolerance and have application as a strategy for inducing long-lived tolerance.

T cells are obtained from the subject to be treated as described above, and a Treg enriched cell population is obtained by negative and or positive selection. An autoantigen-specific regulatory T (Treg) cell enriched composition is one in which the percentage of autoantigen-specific Treg cells is higher than the percentage of autoantigen-specific Treg cells in the originally obtained population of cells. In particular embodiments, at least 75%, 85%, 90%, 95%, or 98% of said cells of the composition are autoantigen-specific regulatory T cells. To maximize efficacy, the subpopulation is enriched to at least 90%, preferably at least 95%, and more preferably at least 98% Treg cells, preferably CD4+CD25+CD62L+ Treg cells. Positive selection may be combined with negative selection against cells comprising surface makers specific to non-Treg cell types, such as depletion of CD8, CD11b, CD16, CD19, CD36 and CD56-bearing cells.

The Treg cells are activated in a polyclonal or antigen-specific manner ex vivo using the compositions, as described above, expanded, and administered to the subject to be treated. In another embodiment, a population of T cells not enriched for Treg cells is activated and expanded, and the Treg cells are selected from the expanded T cell population using appropriate positive and/or negative selection.

Adoptive immunotherapy using Treg cells can be used for prophylactic and therapeutic applications. In prophylactic applications, Treg cells are administered in amounts effective to eliminate or reduce the risk or delay the outset of conditions associated with undesirable activation, over-activation or inappropriate or aberrant activation of an immune response, including physiological, biochemical, histologic and/or behavioral symptoms of the disorder, its complications and intermediate pathological phenotypes presenting during development of the disease or disorder. In therapeutic applications, the compositions and methods disclosed herein are administered to a patient suspected of, or already suffering from such a condition associated with undesirable activation, over-activation or inappropriate or aberrant activation of an immune response to treat, at least partially, the symptoms of the disease (physiological, biochemical, histologic and/or behavioral), including its complications and intermediate pathological phenotypes in development of the disease or disorder. An amount adequate to accomplish therapeutic or prophylactic treatment is defined as a therapeutically- or prophylactically-effective amount.

With respect to allograft rejection or graft versus host disease, in a preferred embodiment, adoptive immunotherapy with Treg cells is initiated prior to transplantation of the allograft. In certain embodiments, the Treg cells can be administered to the subject for a day, three days, a week, two weeks or a month prior to a transplantation. In other embodiments, the Treg cells are administered for a week, two weeks, three weeks, one month, two months, three months or six months following a transplantation. In a preferred embodiment, Treg cells are administered both before and after a transplantation is carried out.

The outcome of the therapeutic and prophylactic methods disclosed herein is to at least produce in a patient a healthful benefit, which includes, but is not limited to, prolonging the lifespan of a patient, delaying the onset of one or more symptoms of the disorder, and/or alleviating a symptom of the disorder after onset of a symptom of the disorder. For example, in the context of allograft rejection, the therapeutic and prophylactic methods can result in prolonging the lifespan of an allograft recipient, prolonging the duration of allograft tolerance prior to rejection, and/or alleviating a symptom associated with allograft rejection.

In another embodiment, undesirable or aberrant antigen-specific immune responses are treated or prevented by adoptive immunotherapy by using the compositions to activate and expand T cells specific for IgE or CD40L.

Immune responses to foreign, sometimes innocuous, substances such as pollen, dust mites, food antigens and bee sting can result in allergic diseases such as hay fever, asthma and systemic anaphylaxis. Immune responses to self-antigens such as pancreatic islet antigens and cartilage antigens can lead to diabetes and arthritis, respectively. The hallmark of the allergic diseases is activation of CD4+ T cells and high production of IgE by B cells, whereas the salient feature of autoimmune diseases are activation of CD4+ T cells and over production of inflammation cytokines. Activated CD4+ T cells transiently express the self antigen CD40L.

Cytotoxic T lymphocytes (CTLs) specific for antigenic peptides derived from IgE molecule can be generated ex vivo using the artificial antigen presenting cells and methods disclosed herein presenting antigenic IgE peptides. These IgE specific CTLs can be administered to a subject to lyse the target cells loaded with IgE peptides and inhibit antigen specific IgE responses in vivo. These IgE specific CTLs can also be used to prevent or treat the development of lung inflammation and airway hypersensitivity.

Similarly, cytotoxic T lymphocytes (CTLs) specific for antigenic peptides derived from CD40L can be generated ex vivo using the artificial antigen presenting cells and methods disclosed herein presenting antigenic CD40L peptides. These CD40L specific CTLs can be administered to a subject to lyse target activated CD4+ cells in vivo. These CD40L specific CTLs can be used to inhibit CD4-dependent antibody responses of all isotypes in vivo.

D. Natural Killer T Cell Immunotherapy

Embodiments disclosed herein can also be used to activate tumor-targeting natural killer T cells (NKT cells). NKT cells are specialized CD1d-restricted T cells that recognize lipid antigens (King et al., Front. Immunol., 9:1519 (2018); Nair & Dhodapkar, Front. Immunol., 8:1178 (2017)). Activated NKT cells promote downstream activation of immune cells within tumors. Unlike other T cells, NKT cells express T cell receptors that recognize lipid antigens presented by CD1d, which is an MHC class I-like molecule. NKT cells also express cytokine receptors, such as those for IL-12, IL-18, IL-25, and IL-23. Activated NKT cells can respond to signals from TCR-mediated stimuli and from inflammatory cytokines by promptly releasing various cytokines, which can in turn affect immune cells present in the tumor microenvironment.

In some embodiments, methods of activating NKT cells employ aAPC substrate particles that are bound to a molecule that is capable of binding endogenous CD1d loaded with antigen. Such substrate particles can be incubated in a lysate prepared from one or more cells of a patient, thereby loading the substrate particles with endogenous CD1d-antigen complexes. These aAPCs can then be used to activate NKT cells, which in some embodiments are NKT type I cells, using methods discussed above for activation of conventional T cells. Embodiments include contacting NKT cells in vivo by administering aAPCs loaded with CD1 d-antigen complexes and contacting NKT cells in vitro and administering activated and proliferated NKT cells in an adoptive immunotherapy approach.

Molecules capable of binding CD1d include various commercially available anti-CD1d antibodies, including but not limited to PA1850 available from Boster; product numbers SAB4301706, SAB1401052, SAB2700866, and HPA072662 available from Sigma Aldrich; and R3G1/51.1, K253, and 1B1 available from BioLegend. Antibodies may be humanized, and may be any variety or derivative of antibodies describe herein or known in the art. CD1d-binding polypeptides can be attached to substrate particles by any method described herein or known in the art.

Cancer antigens and other types of antigens may be targeted by NKT cells activated according to methods disclosed herein. The endogenous CD1d-antigen complexes bound to substrate particles in aAPCs disclosed herein can be derived from a patient's tumor cells or other cancer cells. Endogenous CD1d-antigen complexes may also be derived from antigen presenting cells pulsed with antigen, including, for example, α-GalCer.

### IV. Methods of Making aAPCs

Embodiments disclosed herein include methods of making aAPCs. Such methods may include a steps of contacting a lysate prepared from one or more cells with substrate particles bound to a polypeptide that is capable of specifically binding MHC. The lysate may be prepared from one or more cells obtained from a patient. The cells may be, for example, tumor cells or other cancer cells.

Cells endogenously produce a repertoire of MHC complexes bound to peptides. The repertoire is different for different types of cells. For example, a liver cancer cell will produce cancer-specific antigens that are not produced by normal tissue cells. Preparing a lysate of cells results in endogenous MHC-peptide complexes being released into the lysate, which can then be pulled down by incubating substrate particles in the lysate under conditions in which the substrate particles can become bound to endogenous MHC-peptide complexes. Substrate particles can become bound to the MHC-peptide complexes if, for example, the substrate particles are bound to a polypeptide that is capable of binding endogenous MHC-peptide complexes, of which many examples are provided above. Persons of ordinary skill in the art will know how to adjust conditions, such as buffer concentrations, salt concentrations, detergents, surfactants, temperature, and other parameters that affect the ability of an MHC-binding polypeptide to bind to the endogenous MHC in the lysate.

Lysate may be prepared from a population of cells obtained from a patient to be treated using the aAPC produced using the lysate. The population of cells may be disease cells, such as cancer cells, that endogenously produce MHC complexes bound to peptides from antigens associated with the disease. The population of cells may be obtained from the patient by, for example, taking a biopsy of a tumor or a blood sample from a patient having a blood cancer.

Lysate used in methods of making aAPCs may also be prepared from antigen presenting cells, such as dendritic cells, that have been pulsed with a target antigen. The antigen presenting cells pulsed with antigen endogenously produce MHC complexes bound to peptides from the target antigen, which can then be pulled down from a lysate of the antigen presenting cells as described above.

Preparation of lysate can be performed by a number of methods known in the art. The conditions for lysis should be chosen to preserve interactions among MHC complex subunits and between the MHC complex and antigen peptides. Lysate can be prepared, for example, by mechanical means, by bead beating, by freezing and thawing, by sonication, by pulverization, by use of detergents, or by enzymatic digestion using, for example, hyaluronidase, dispase, proteases, and nucleases.

The endogenous MHC-peptide complexes can be pulled down directly from lysate, with lysate being used to refer to the composition obtained when cells are lysed and optionally the cellular debris (e.g., cellular membranes) is removed. In some embodiments, one or more purification and/or fractionation steps are performed before the MHC-peptide complexes are pulled down. The composition resulting from such purification and/or fractionation is not referred to herein as lysate. In embodiments in which fractionation and/or purification steps are performed, the conditions are chosen such that MHC-peptide complexes remain intact.

### V. Treating Cancer

The methods of the disclosure may be used to treat a cancer. The cancers amenable for treatment may include, but are not limited to, tumors of all types, locations, sizes, and characteristics. The methods and compositions of the disclosure are suitable for treating, for example, pancreatic cancer, colon cancer, acute myeloid leukemia, adrenocortical carcinoma, AIDS-related cancers, AIDS-related lymphoma, anal cancer, appendix cancer, astrocytoma, childhood cerebellar or cerebral basal cell carcinoma, bile duct cancer, extrahepatic bladder cancer, bone cancer, osteosarcoma/malignant fibrous histiocytoma, brainstem glioma, brain tumor, cerebellar astrocytoma brain tumor, cerebral astrocytoma/malignant glioma brain tumor, ependymoma brain tumor, medulloblastoma brain tumor, supratentorial primitive neuroectodermal tumors brain tumor, visual pathway and hypothalamic glioma, breast cancer, specific breast cancers such as ductal carcinoma in situ, invasive ductal carcinoma, tubular carcinoma of the breast, medullary carcinoma of the breast, mucinous carcinoma of the breast, papillary carcinoma of the breast, cribriform carcinoma of the breast, invasive lobular carcinoma, inflammatory breast cancer, lobular carcinoma in situ, male breast cancer, Paget's disease of the nipple, phyllodes tumors of the breast, recurrent and/or metastatic breast, cancer, luminal A or B breast cancer, triple-negative/basal-like breast cancer, and HER2-enriched breast cancer, lymphoid cancer, bronchial adenomas/carcinoids, tracheal cancer, Burkitt lymphoma, carcinoid tumor, childhood carcinoid tumor, gastrointestinal carcinoma of unknown primary, central nervous system lymphoma, primary cerebellar astrocytoma, childhood cerebral astrocytoma/malignant glioma, childhood cervical cancer, childhood cancers, chronic lymphocytic leukemia, chronic myelogenous leukemia, chronic myeloproliferative disorders, cutaneous T-cell lymphoma, desmoplastic small round cell tumor, endometrial cancer, ependymoma, esophageal cancer, Ewing's, childhood extragonadal Germ cell tumor, extrahepatic bile duct cancer, eye cancer, retinoblastoma, gallbladder cancer, gastric (stomach) cancer, gastrointestinal carcinoid tumor, gastrointestinal stromal tumor (GIST), germ cell tumor: extracranial, extragonadal, or ovarian, gestational trophoblastic tumor, glioma of the brain stem, glioma, childhood cerebral astrocytoma, childhood visual pathway and hypothalamic glioma, gastric carcinoid, hairy cell leukemia, head and neck cancer, heart cancer, hepatocellular (liver) cancer, Hodgkin lymphoma, hypopharyngeal cancer, hypothalamic and visual pathway glioma, childhood intraocular melanoma, islet cell carcinoma (endocrine pancreas), kaposi sarcoma, kidney cancer (renal cell cancer), laryngeal cancer, leukemia, acute lymphoblastic (also called acute lymphocytic leukemia) leukemia, acute myeloid (also called acute myelogenous leukemia) leukemia, chronic lymphocytic (also called chronic lymphocytic leukemia) leukemia, chronic myelogenous (also called chronic myeloid leukemia) leukemia, hairy cell lip and oral cavity cancer, liposarcoma, liver cancer (primary), non-small cell lung cancer, small cell lung cancer, lymphomas, AIDS-related lymphoma, Burkitt lymphoma, cutaneous T-cell lymphoma, Hodgkin lymphoma, Non-Hodgkin (an old classification of all lymphomas except Hodgkin's) lymphoma, primary central nervous system lymphoma, Waldenstrom macroglobulinemia, malignant fibrous histiocytoma of bone/osteosarcoma, childhood medulloblastoma, intraocular (eye) melanoma, merkel cell carcinoma, adult malignant mesothelioma, childhood mesothelioma, metastatic squamous neck cancer, mouth cancer, multiple endocrine neoplasia syndrome, multiple myeloma/plasma cell neoplasm, mycosis fungoides, myelodysplastic syndromes, myelodysplastic/myeloproliferative diseases, chronic myelogenous leukemia, adult acute myeloid leukemia, childhood acute myeloid leukemia, multiple myeloma, chronic myeloproliferative disorders, nasal cavity and paranasal sinus cancer, nasopharyngeal carcinoma, neuroblastoma, oral cancer, oropharyngeal cancer, osteosarcoma/malignant, fibrous histiocytoma of bone, ovarian cancer, ovarian epithelial cancer (surface epithelial-stromal tumor), ovarian germ cell tumor, ovarian low malignant potential tumor, pancreatic cancer, islet cell paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors, childhood pituitary adenoma, plasma cell neoplasia/multiple myeloma, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma, childhood Salivary gland cancer Sarcoma, Ewing family of tumors, Kaposi sarcoma, soft tissue sarcoma, uterine sezary syndrome sarcoma, skin cancer (nonmelanoma), skin cancer (melanoma), skin carcinoma, Merkel cell small cell lung cancer, small intestine cancer, soft tissue sarcoma, squamous cell carcinoma. squamous neck cancer with occult primary, metastatic stomach cancer, supratentorial primitive neuroectodermal tumor, childhood T-cell lymphoma, testicular cancer, throat cancer, thymoma, childhood thymoma, thymic carcinoma, thyroid cancer, urethral cancer, uterine cancer, endometrial uterine sarcoma, vaginal cancer, visual pathway and hypothalamic glioma, childhood vulvar cancer, and wilms tumor (kidney cancer).

### VI. Administering Treatment

Embodiments of the disclosure relate to compositions and methods comprising therapeutic compositions and pharmaceutical compositions and formulations. Therapeutic compositions, pharmaceutical compositions, and pharmaceutical formulations may include, for example, aAPCs or compositions comprising aAPCs. aAPCs are referred to herein as “therapeutic agents.” The different therapies may be administered in one composition or in more than one composition, such as 2 compositions, 3 compositions, or 4 compositions. Various combinations of the agents may be employed.

The therapeutic agents of the disclosure may be administered by the same route of administration or by different routes of administration. In some embodiments, the therapy is administered intravenously, intramuscularly, subcutaneously, topically, orally, transdermally, intraperitoneally, intraorbitally, by implantation, by inhalation, intrathecally, intraventricularly, or intranasally. In some embodiments, the antibiotic is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

The compounds utilized in the compositions and methods of this invention can also be modified by appending appropriate functionalities to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

According to a preferred embodiment, the compositions of this invention are formulated for pharmaceutical administration to a mammal, preferably a human being.

Sterile injectable forms of the compositions of this invention can be aqueous or oleaginous suspension. These suspensions can be formulated according to techniques known in the art using suitable dispersing or wetting agents and suspending agents. The sterile injectable preparation can also be a sterile injectable solution or suspension in a non-toxic parenterally acceptable diluent or solvent. Among the acceptable vehicles and solvents that can be employed are water, Ringer's solution, phosphate buffer saline (PBS), and isotonic sodium chloride solution. In addition, sterile, fixed oils are conventionally employed as a solvent or suspending medium. Fatty acids, such as oleic acid and its glyceride derivatives are useful in the preparation of injectables, as are natural pharmaceutically-acceptable oils, such as olive oil and castor oil, especially in their polyoxyethylated versions. These oil solutions or suspensions can also contain a long-chain alcohol diluent or dispersant, such as carboxymethyl cellulose or similar dispersing agents which are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms can also be used for the purposes of formulation.

If a solid carrier is used, the preparation can be tableted, placed in a hard gelating capsule in powder or pellet form, or in the form of a troche or lozenge. The amount of solid carrier will vary, e.g., from about 25 mg to 400 mg. When a liquid carrier is used, the preparation can be, e.g., in the form of a syrup, emulsion, soft gelatin capsule, sterile injectable liquid such as an ampule or nonaqueous liquid suspension. Where the composition is in the form of a capsule, any routine encapsulation is suitable, for example, using the aforementioned carriers in a hard gelatin capsule shell.

A syrup formulation can consist of a suspension or solution of the compound in a liquid carrier for example, ethanol, glycerine, or water with a flavoring or coloring agent. An aerosol preparation can consist of a solution or suspension of the compound in a liquid carrier such as water, ethanol or glycerine; whereas in a powder dry aerosol, the preparation can include e.g., a wetting agent.

Formulations of the present invention comprise an active ingredient together with one or more acceptable carrier(s) thereof and optionally any other therapeutic ingredient(s). The carrier(s) should be “acceptable” in the sense of being compatible with the other ingredients of the formulation and not deleterious to the recipient thereof.

The pharmaceutical compositions of this invention can be orally administered in any orally acceptable dosage form including, but not limited to, capsules, tablets, and aqueous suspensions or solutions. In the case of tablets for oral use, carriers that are commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents can also be added.

It will be recognized by one of skill in the art that the form and character of the pharmaceutically acceptable carrier or diluent is dictated by the amount of active ingredient or therapeutic agent with which it is to be combined, the route of administration, and other well-known variables.

Upon improvement of a patient's condition, a maintenance dose of a compound, composition or combination of this invention can be administered, if necessary. Subsequently, the dosage or frequency of administration, or both, can be reduced, as a function of the symptoms, to a level at which the improved condition is retained when the symptoms have been alleviated to the desired level, treatment should cease. Patients can, however, require intermittent treatment on a long-term basis upon any recurrence or disease symptoms.

In general, the aAPCs described herein are useful for treating a subject having or being predisposed to any disease or disorder to which the subjects immune system mounts an immune response. Treating a disease or disorder to which the subject's immune system mounts an immune response may include inhibiting or delaying the development of the disease or disorder or inhibiting or reducing the symptoms of the disease or disorder. The compositions are useful as prophylactic compositions, which confer resistance in a subject to subsequent tumor development or exposure to infectious agents. The compositions are also useful as therapeutic compositions, which can be used to initiate or enhance a subject's immune response to a pre-existing antigen, such as a tumor antigen in a subject with cancer, or a viral antigen in a subject infected with a virus.

The compositions are also useful to treat or prevent diseases and disorders characterized by undesirable activation, overactivation or inappropriate activation of the immune system, such as occurs during allergic responses, autoimmune diseases and disorders, graft rejection and graft-versus-host-disease. Methods for using aAPCs for treatment of these conditions is described in more detail below.

The ability of the aAPCs to elicit T-cell mediated immune responses by activation and expansion of T cells makes these compositions especially useful for eliciting a cell-mediated response to a disease-related antigen in order to attack the disease. Thus, in a preferred embodiment, the type of disease to be treated or prevented is a malignant tumor or a chronic infectious disease caused by a bacterium, virus, protozoan, helminth, or other microbial pathogen that enters intracellularly and is attacked, i.e., by the cytotoxic T lymphocytes.

The desired outcome of a prophylactic, therapeutic or de-sensitized immune response may vary according to the disease, according to principles well known in the art. For example, an immune response against an infectious agent may completely prevent colonization and replication of an infectious agent, affecting “sterile immunity” and the absence of any disease symptoms. However, treatment against infectious agents with aAPCs may be considered effective if it reduces the number, severity or duration of symptoms; if it reduces the number of individuals in a population with symptoms; or reduces the transmission of an infectious agent. Similarly, immune responses against cancer, allergens or infectious agents may completely treat a disease, may alleviate symptoms, or may be one facet in an overall therapeutic intervention against a disease. For example, the stimulation of an immune response against a cancer may be coupled with surgical, chemotherapeutic, radiologic, hormonal and other immunologic approaches in order to affect treatment.

In some instances, the subject can be treated prophylactically, such as when there may be a risk of developing disease from an infectious agent. Infectious agents include bacteria, viruses and parasites. An individual traveling to or living in an area of endemic infectious disease may be considered to be at risk and a candidate for prophylactic vaccination against the particular infectious agent. Preventative treatment can be applied to any number of diseases where there is a known relationship between the particular disease and a particular risk factor, such as geographical location or work environment.

Subjects to be treated using methods disclosed herein include in some embodiments subjects with or with a risk of developing malignant tumors. In a mature animal, a balance usually is maintained between cell renewal and cell death in most organs and tissues. The various types of mature cells in the body have a given life span; as these cells die, new cells are generated by the proliferation and differentiation of various types of stem cells. Under normal circumstances, the production of new cells is so regulated that the numbers of any particular type of cell remain constant. Occasionally, though, cells arise that are no longer responsive to normal growth-control mechanisms. These cells give rise to clones of cells that can expand to a considerable size, producing a tumor or neoplasm. A tumor that is not capable of indefinite growth and does not invade the healthy surrounding tissue extensively is benign. A tumor that continues to grow and becomes progressively invasive is malignant. The term cancer refers specifically to a malignant tumor. In addition to uncontrolled growth, malignant tumors exhibit metastasis. In this process, small clusters of cancerous cells dislodge from a tumor, invade the blood or lymphatic vessels, and are carried to other tissues, where they continue to proliferate. In this way a primary tumor at one site can give rise to a secondary tumor at another site. The compositions and method described herein may be useful for treating subjects having malignant tumors. Treating a subject having a malignant tumor includes delaying or inhibiting the growth of a tumor in a subject, reducing the growth or size of the tumor, inhibiting or reducing metastasis of the tumor, and inhibiting or reducing symptoms associated with tumor development or growth. For instance, the examples below demonstrate that the aAPCs disclosed herein are effective in significantly delaying the growth of tumors in vivo.

Malignant tumors which may be treated are classified herein according to the embryonic origin of the tissue from which the tumor is derived. Carcinomas are tumors arising from endodermal or ectodermal tissues such as skin or the epithelial lining of internal organs and glands. A melanoma is a type of carcinoma of the skin for which this invention is particularly useful. Sarcomas, which arise less frequently, are derived from mesodermal connective tissues such as bone, fat, and cartilage. The leukemias and lymphomas are malignant tumors of hematopoietic cells of the bone marrow. Leukemias proliferate as single cells, whereas lymphomas tend to grow as tumor masses. Malignant tumors may show up at numerous organs or tissues of the body to establish a cancer.

The types of cancer that can be treated in with the provided compositions and methods include, but are not limited to, the following: bladder, brain, breast, cervical, colo rectal, esophageal, kidney, liver, lung, nasopharyngeal, pancreatic, prostate, skin, stomach, uterine, and the like. Administration is not limited to the treatment of an existing tumor or infectious disease but can also be used to prevent or lower the risk of developing such diseases in an individual, i.e., for prophylactic use. Potential candidates for prophylactic vaccination include individuals with a high risk of developing cancer, i.e., with a personal or familial history of certain types of cancer.

AAPCs can also be used in embodiments disclosed herein for treatment of disease conditions characterized by immunosuppression, including, but not limited to, AIDS or AIDS-related complex, idiopathic immunosuppression, drug induced immunosuppression, other virally or environmentally-induced conditions, and certain congenital immune deficiencies. AAPCs can also be employed to increase immune function that has been impaired by the use of radiotherapy of immunosuppressive drugs (e.g., certain chemotherapeutic agents), and therefore can be particularly useful when used in conjunction with such drugs or radiotherapy.

Embodiments of compositions and methods disclosed herein are also useful to treat and/or preventing allergic reactions, such as allergic reactions which lead to anaphylaxis. Allergic reactions may be characterized by the TH2 responses against an antigen leading to the presence of IgE antibodies. Stimulation of TH1 immune responses and the production of IgG antibodies may alleviate allergic disease. Thus, the disclosed vaccine compositions may lead to the production of antibodies that prevent and/or attenuate allergic reactions in subjects exposed to allergens. These can be used to enhance blocking or tolerance inducing reactions.

Embodiments of compositions and methods disclosed herein are useful for the treatment or prevention of autoimmune diseases and disorders. Exemplary autoimmune diseases include vasculitis, Wegener's granulomatosis, Addison's disease, alopecia, ankylosing spondylitis, antiphospholipid syndrome, Behcet's disease, celiac disease, chronic fatigue syndrome, Crohn's disease, ulcerative colitis, type I diabetes, fibromyalgia, autoimmune gastritis, Goodpasture syndrome, Graves' disease, idiopathic thrombocytopenic purpura (ITP), lupus, Meniere's multiple sclerosis, myasthenia gravis, pemphigus vulgaris, primary biliary cirrhosis, psoriasis, rheumatoid arthritis, rheumatic fever, sarcoidosis, scleroderma, vitiligo, vasculitis, small vessel vasculitis, hepatitis, primary biliary cirrhosis, rheumatoid arthritis, Crohn's disease, ulcerative colitis, sarcoidosis, scleroderma, graft versus host disease (acute and chronic), aplastic anemia, and cyclic neutropenia.

Embodiments of compositions and methods disclosed herein are useful for the treatment or prevention of graft rejection or graft versus host disease. The methods and compositions can be used in the prevention or treatment of any type of allograft rejection or graft versus host disease for any type of graft, including a xenograft. The allograft can be an organ transplant, such as, but not limited to, a heart, kidney, liver, lung or pancreas. Alternatively, the allograft can be a tissue transplant, such as, but not limited to, heart valve, endothelial, cornea, eye lens or bone marrow tissue transplant. In yet other embodiments, the allograft can be a skin graft.

### VII. Additional Therapies

Embodiments of the disclosure relate to the administration of an additional therapy. In some embodiments, the additional therapy comprises oncolytic virus, polysaccharide, neoantigen, chemotherapy, radiotherapy, surgery, or other therapy described below or throughout the disclosure.

A. Checkpoint Inhibitors and Combination Treatment

Embodiments of the disclosure may include administration of immune checkpoint inhibitors (also referred to as checkpoint inhibitor therapy), which are further described below.

1. PD-1, PDL1, and PDL2 Inhibitors

PD-1 can act in the tumor microenvironment where T cells encounter an infection or tumor. Activated T cells upregulate PD-1 and continue to express it in the peripheral tissues. Cytokines such as IFN-gamma induce the expression of PDL1 on epithelial cells and tumor cells. PDL2 is expressed on macrophages and dendritic cells. The main role of PD-1 is to limit the activity of effector T cells in the periphery and prevent excessive damage to the tissues during an immune response. Inhibitors of the disclosure may block one or more functions of PD-1 and/or PDL1 activity.

Alternative names for “PD-1” include CD279 and SLEB2. Alternative names for “PDL1” include B7-H1, B7-4, CD274, and B7-H. Alternative names for “PDL2” include B7-DC, Btdc, and CD273. In some embodiments, PD-1, PDL1, and PDL2 are human PD-1, PDL1 and PDL2.

In some embodiments, the PD-1 inhibitor is a molecule that inhibits the binding of PD-1 to its ligand binding partners. In a specific aspect, the PD-1 ligand binding partners are PDL1 and/or PDL2. In another embodiment, a PDL1 inhibitor is a molecule that inhibits the binding of PDL1 to its binding partners. In a specific aspect, PDL1 binding partners are PD-1 and/or B7-1. In another embodiment, the PDL2 inhibitor is a molecule that inhibits the binding of PDL2 to its binding partners. In a specific aspect, a PDL2 binding partner is PD-1. The inhibitor may be an antibody, an antigen binding fragment thereof, an immunoadhesin, a fusion protein, or oligopeptide. Exemplary antibodies are described in U.S. Pat. Nos. 8,735,553, 8,354,509, and 8,008,449, all incorporated herein by reference. Other PD-1 inhibitors for use in the methods and compositions provided herein are known in the art such as described in U.S. Patent Application Nos. US2014/0294898, US2014/022021, and US2011/0008369, all incorporated herein by reference.

In some embodiments, the PD-1 inhibitor is an anti-PD-1 antibody (e.g., a human antibody, a humanized antibody, or a chimeric antibody). In some embodiments, the anti-PD-1 antibody is selected from the group consisting of nivolumab, pembrolizumab, and pidilizumab. In some embodiments, the PD-1 inhibitor is an immunoadhesin (e.g., an immunoadhesin comprising an extracellular or PD-1 binding portion of PDL1 or PDL2 fused to a constant region (e.g., an Fc region of an immunoglobulin sequence). In some embodiments, the PDL1 inhibitor comprises AMP-224. Nivolumab, also known as MDX-1106-04, MDX-1106, ONO-4538, BMS-936558, and OPDIVO®, is an anti-PD-1 antibody described in WO2006/121168. Pembrolizumab, also known as MK-3475, Merck 3475, lambrolizumab, KEYTRUDA®, and SCH-900475, is an anti-PD-1 antibody described in WO2009/114335. Pidilizumab, also known as CT-011, hBAT, or hBAT-1, is an anti-PD-1 antibody described in WO2009/101611. AMP-224, also known as B7-DCIg, is a PDL2-Fc fusion soluble receptor described in WO2010/027827 and WO2011/066342. Additional PD-1 inhibitors include MEDI0680, also known as AMP-514, and REGN2810.

In some embodiments, the immune checkpoint inhibitor is a PDL1 inhibitor such as Durvalumab, also known as MEDI4736, atezolizumab, also known as MPDL3280A, avelumab, also known as MSB00010118C, MDX-1105, BMS-936559, or combinations thereof. In certain aspects, the immune checkpoint inhibitor is a PDL2 inhibitor such as rHIgM12B7.

In some embodiments, the inhibitor comprises the heavy and light chain CDRs or VRs of nivolumab, pembrolizumab, or pidilizumab. Accordingly, in one embodiment, the inhibitor comprises the CDR1, CDR2, and CDR3 domains of the VH region of nivolumab, pembrolizumab, or pidilizumab, and the CDR1, CDR2 and CDR3 domains of the VL region of nivolumab, pembrolizumab, or pidilizumab. In another embodiment, the antibody competes for binding with and/or binds to the same epitope on PD-1, PDL1, or PDL2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

2. CTLA-4, B7-1, and B7-2

Another immune checkpoint that can be targeted in the methods provided herein is the cytotoxic T-lymphocyte-associated protein 4 (CTLA-4), also known as CD152. The complete cDNA sequence of human CTLA-4 has the Genbank accession number L15006. CTLA-4 is found on the surface of T cells and acts as an “off” switch when bound to B7-1 (CD80) or B7-2 (CD86) on the surface of antigen-presenting cells. CTLA4 is a member of the immunoglobulin superfamily that is expressed on the surface of Helper T cells and transmits an inhibitory signal to T cells. CTLA4 is similar to the T-cell co-stimulatory protein, CD28, and both molecules bind to B7-1 and B7-2 on antigen-presenting cells. CTLA-4 transmits an inhibitory signal to T cells, whereas CD28 transmits a stimulatory signal. Intracellular CTLA-4 is also found in regulatory T cells and may be important to their function. T cell activation through the T cell receptor and CD28 leads to increased expression of CTLA-4, an inhibitory receptor for B7 molecules. Inhibitors of the disclosure may block one or more functions of CTLA-4, B7-1, and/or B7-2 activity. In some embodiments, the inhibitor blocks the CTLA-4 and B7-1 interaction. In some embodiments, the inhibitor blocks the CTLA-4 and B7-2 interaction.

In some embodiments, the immune checkpoint inhibitor is an anti-CTLA-4 antibody (e.g., a human antibody, a humanized antibody, or a chimeric antibody), an antigen binding fragment thereof, an immunoadhesin, a fusion protein, or oligopeptide.

Anti-human-CTLA-4 antibodies (or VH and/or VL domains derived therefrom) suitable for use in the present methods can be generated using methods well known in the art. Alternatively, art recognized anti-CTLA-4 antibodies can be used. For example, the anti-CTLA-4 antibodies disclosed in: U.S. Pat. No. 8,119,129, WO 01/14424, WO 98/42752; WO 00/37504 (CP675,206, also known as tremelimumab; formerly ticilimumab), U.S. Pat. No. 6,207,156; Hurwitz et al., 1998; can be used in the methods disclosed herein. The teachings of each of the aforementioned publications are hereby incorporated by reference. Antibodies that compete with any of these art-recognized antibodies for binding to CTLA-4 also can be used. For example, a humanized CTLA-4 antibody is described in International Patent Application No. WO2001/014424, WO2000/037504, and U.S. Pat. No. 8,017,114; all incorporated herein by reference.

A further anti-CTLA-4 antibody useful as a checkpoint inhibitor in the methods and compositions of the disclosure is ipilimumab (also known as 10D1, MDX-010, MDX-101, and Yervoy®) or antigen binding fragments and variants thereof (see, e.g., WOO 1/14424).

In some embodiments, the inhibitor comprises the heavy and light chain CDRs or VRs of tremelimumab or ipilimumab. Accordingly, in one embodiment, the inhibitor comprises the CDR1, CDR2, and CDR3 domains of the VH region of tremelimumab or ipilimumab, and the CDR1, CDR2 and CDR3 domains of the VL region of tremelimumab or ipilimumab. In another embodiment, the antibody competes for binding with and/or binds to the same epitope on PD-1, B7-1, or B7-2 as the above-mentioned antibodies. In another embodiment, the antibody has at least about 70, 75, 80, 85, 90, 95, 97, or 99% (or any derivable range therein) variable region amino acid sequence identity with the above-mentioned antibodies.

B. CAR-T Cell Therapy

Chimeric antigen receptors (CARs, also known as chimeric immunoreceptors, chimeric T cell receptors or artificial T cell receptors) are engineered receptors that combine a new specificity with an immune cell to target cancer cells. Typically, these receptors graft the specificity of a monoclonal antibody onto a T cell. The receptors are called chimeric because they are fused of parts from different sources. CAR-T cell therapy refers to a treatment that uses such transformed cells for cancer therapy.

The basic principle of CAR-T cell design involves recombinant receptors that combine antigen-binding and T-cell activating functions. The general premise of CAR-T cells is to artificially generate T-cells targeted to markers found on cancer cells. Scientists can remove T-cells from a person, genetically alter them, and put them back into the patient for them to attack the cancer cells. Once the T cell has been engineered to become a CAR-T cell, it acts as a “living drug”. CAR-T cells create a link between an extracellular ligand recognition domain to an intracellular signaling molecule which in turn activates T cells. The extracellular ligand recognition domain is usually a single-chain variable fragment (scFv). An important aspect of the safety of CAR-T cell therapy is how to ensure that only cancerous tumor cells are targeted, and not normal cells. The specificity of CAR-T cells is determined by the choice of molecule that is targeted.

Exemplary CAR-T therapies include Tisagenlecleucel (Kymriah) and Axicabtagene ciloleucel (Yescarta). In some embodiments, the CAR-T therapy targets CD19.

C. Cytokine Therapy

Cytokines are proteins produced by many types of cells present within a tumor. They can modulate immune responses. The tumor often employs them to allow it to grow and reduce the immune response. These immune-modulating effects allow them to be used as drugs to provoke an immune response. Two commonly used cytokines are interferons and interleukins.

Interferons are produced by the immune system. They are usually involved in anti-viral response, but also have use for cancer. They fall in three groups: type I (IFNα and IFNβ), type II (IFNγ) and type III (IFNλ).

Interleukins have an array of immune system effects. IL-2 is an exemplary interleukin cytokine therapy.

D. Oncolytic Virus

In some embodiments, the additional therapy comprises an oncolytic virus. An oncolytic virus is a virus that preferentially infects and kills cancer cells. As the infected cancer cells are destroyed by oncolysis, they release new infectious virus particles or virions to help destroy the remaining tumor. Oncolytic viruses are thought not only to cause direct destruction of the tumor cells, but also to stimulate host anti-tumor immune responses for long-term immunotherapy.

E. Polysaccharides

In some embodiments, the additional therapy comprises polysaccharides. Certain compounds found in mushrooms, primarily polysaccharides, can up-regulate the immune system and may have anti-cancer properties. For example, beta-glucans such as lentinan have been shown in laboratory studies to stimulate macrophage, NK cells, T cells and immune system cytokines and have been investigated in clinical trials as immunologic adjuvants.

F. Neoantigens

In some embodiments, the additional therapy comprises neoantigen administration. Many tumors express mutations. These mutations potentially create new targetable antigens (neoantigens) for use in T cell immunotherapy. The presence of CD8+ T cells in cancer lesions, as identified using RNA sequencing data, is higher in tumors with a high mutational burden. The level of transcripts associated with cytolytic activity of natural killer cells and T cells positively correlates with mutational load in many human tumors.

G. Chemotherapies

In some embodiments, the additional therapy comprises a chemotherapy. Suitable classes of chemotherapeutic agents include (a) Alkylating Agents, such as nitrogen mustards (e.g., mechlorethamine, cylophosphamide, ifosfamide, melphalan, chlorambucil), ethylenimines and methylmelamines (e.g., hexamethylmelamine, thiotepa), alkyl sulfonates (e.g., busulfan), nitrosoureas (e.g., carmustine, lomustine, chlorozoticin, streptozocin) and triazines (e.g., dicarbazine), (b) Antimetabolites, such as folic acid analogs (e.g., methotrexate), pyrimidine analogs (e.g., 5-fluorouracil, floxuridine, cytarabine, azauridine) and purine analogs and related materials (e.g., 6-mercaptopurine, 6-thioguanine, pentostatin), (c) Natural Products, such as vinca alkaloids (e.g., vinblastine, vincristine), epipodophylotoxins (e.g., etoposide, teniposide), antibiotics (e.g., dactinomycin, daunorubicin, doxorubicin, bleomycin, plicamycin and mitoxanthrone), enzymes (e.g., L-asparaginase), and biological response modifiers (e.g., Interferon-α), and (d) Miscellaneous Agents, such as platinum coordination complexes (e.g., cisplatin, carboplatin), substituted ureas (e.g., hydroxyurea), methylhydiazine derivatives (e.g., procarbazine), and adreocortical suppressants (e.g., taxol and mitotane). In some embodiments, cisplatin is a particularly suitable chemotherapeutic agent.

Cisplatin has been widely used to treat cancers such as, for example, metastatic testicular or ovarian carcinoma, advanced bladder cancer, head or neck cancer, cervical cancer, lung cancer or other tumors. Cisplatin is not absorbed orally and must therefore be delivered via other routes such as, for example, intravenous, subcutaneous, intratumoral or intraperitoneal injection. Cisplatin can be used alone or in combination with other agents, with efficacious doses used in clinical applications including about 15 mg/m² to about 20 mg/m² for 5 days every three weeks for a total of three courses being contemplated in certain embodiments. In some embodiments, the amount of cisplatin delivered to the cell and/or subject in conjunction with the construct comprising an Egr-1 promoter operatively linked to a polynucleotide encoding the therapeutic polypeptide is less than the amount that would be delivered when using cisplatin alone.

Other suitable chemotherapeutic agents include antimicrotubule agents, e.g., Paclitaxel (“Taxol”) and doxorubicin hydrochloride (“doxorubicin”). The combination of an Egr-1 promoter/TNFα construct delivered via an adenoviral vector and doxorubicin was determined to be effective in overcoming resistance to chemotherapy and/or TNF-α, which suggests that combination treatment with the construct and doxorubicin overcomes resistance to both doxorubicin and TNF-α.

Doxorubicin is absorbed poorly and is preferably administered intravenously. In certain embodiments, appropriate intravenous doses for an adult include about 60 mg/m² to about 75 mg/m² at about 21-day intervals or about 25 mg/m² to about 30 mg/m² on each of 2 or 3 successive days repeated at about 3 week to about 4 week intervals or about 20 mg/m² once a week. The lowest dose should be used in elderly patients, when there is prior bone-marrow depression caused by prior chemotherapy or neoplastic marrow invasion, or when the drug is combined with other myelopoietic suppressant drugs.

Nitrogen mustards are another suitable chemotherapeutic agent useful in the methods of the disclosure. A nitrogen mustard may include, but is not limited to, mechlorethamine (HN₂), cyclophosphamide and/or ifosfamide, melphalan (L-sarcolysin), and chlorambucil. Cyclophosphamide (CYTOXAN®) is available from Mead Johnson and NEOSTAR® is available from Adria), is another suitable chemotherapeutic agent. Suitable oral doses for adults include, for example, about 1 mg/kg/day to about 5 mg/kg/day, intravenous doses include, for example, initially about 40 mg/kg to about 50 mg/kg in divided doses over a period of about 2 days to about 5 days or about 10 mg/kg to about 15 mg/kg about every 7 days to about 10 days or about 3 mg/kg to about 5 mg/kg twice a week or about 1.5 mg/kg/day to about 3 mg/kg/day. Because of adverse gastrointestinal effects, the intravenous route is preferred. The drug also sometimes is administered intramuscularly, by infiltration or into body cavities.

Additional suitable chemotherapeutic agents include pyrimidine analogs, such as cytarabine (cytosine arabinoside), 5-fluorouracil (fluouracil; 5-FU) and floxuridine (fluorode-oxyuridine; FudR). 5-FU may be administered to a subject in a dosage of anywhere between about 7.5 to about 1000 mg/m². Further, 5-FU dosing schedules may be for a variety of time periods, for example up to six weeks, or as determined by one of ordinary skill in the art to which this disclosure pertains.

Gemcitabine diphosphate (GEMZAR®, Eli Lilly & Co., “gemcitabine”), another suitable chemotherapeutic agent, is recommended for treatment of advanced and metastatic pancreatic cancer, and will therefore be useful in the present disclosure for these cancers as well.

The amount of the chemotherapeutic agent delivered to the patient may be variable. In one suitable embodiment, the chemotherapeutic agent may be administered in an amount effective to cause arrest or regression of the cancer in a host, when the chemotherapy is administered with the construct. In other embodiments, the chemotherapeutic agent may be administered in an amount that is anywhere between 2 to 10,000 fold less than the chemotherapeutic effective dose of the chemotherapeutic agent. For example, the chemotherapeutic agent may be administered in an amount that is about 20 fold less, about 500 fold less or even about 5000 fold less than the chemotherapeutic effective dose of the chemotherapeutic agent. The chemotherapeutics of the disclosure can be tested in vivo for the desired therapeutic activity in combination with the construct, as well as for determination of effective dosages. For example, such compounds can be tested in suitable animal model systems prior to testing in humans, including, but not limited to, rats, mice, chicken, cows, monkeys, rabbits, etc. In vitro testing may also be used to determine suitable combinations and dosages, as described in the examples.

H. Radiotherapy

In some embodiments, the additional therapy or prior therapy comprises radiation, such as ionizing radiation. As used herein, “ionizing radiation” means radiation comprising particles or photons that have sufficient energy or can produce sufficient energy via nuclear interactions to produce ionization (gain or loss of electrons). An exemplary and preferred ionizing radiation is an x-radiation. Means for delivering x-radiation to a target tissue or cell are well known in the art.

In some embodiments, the amount of ionizing radiation is greater than 20 Gy and is administered in one dose. In some embodiments, the amount of ionizing radiation is 18 Gy and is administered in three doses. In some embodiments, the amount of ionizing radiation is at least, at most, or exactly 2, 4, 6, 8, 10, 15, 16, 17, 18, 19, 20, 21, 22, 23, 24, 25, 26, 27, 18, 19, 30, 31, 32, 33, 34, 35, 36, 37, 38, 39, 40, 41, 42, 43, 44, 45, 46, 47, 48, 49, or 40 Gy (or any derivable range therein). In some embodiments, the ionizing radiation is administered in at least, at most, or exactly 1, 2, 3, 4, 5, 6, 7, 8, 9, or 10 does (or any derivable range therein). When more than one dose is administered, the does may be about 1, 4, 8, 12, or 24 hours or 1, 2, 3, 4, 5, 6, 7, or 8 days or 1, 2, 3, 4, 5, 6, 7, 8, 9, 10, 12, 14, or 16 weeks apart, or any derivable range therein.

In some embodiments, the amount of IR may be presented as a total dose of IR, which is then administered in fractionated doses. For example, in some embodiments, the total dose is 50 Gy administered in 10 fractionated doses of 5 Gy each. In some embodiments, the total dose is 50-90 Gy, administered in 20-60 fractionated doses of 2-3 Gy each. In some embodiments, the total dose of IR is at least, at most, or about 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 125, 130, 135, 140, or 150 (or any derivable range therein). In some embodiments, the total dose is administered in fractionated doses of at least, at most, or exactly 1, 2, 3, 4, 5, 6, 7, 8, 9, 10, 12, 14, 15, 20, 25, 30, 35, 40, 45, or 50 Gy (or any derivable range therein. In some embodiments, at least, at most, or exactly 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 fractionated doses are administered (or any derivable range therein). In some embodiments, at least, at most, or exactly 1, 2, 3, 4, 5, 6, 7, 8, 9, 10, 11, or 12 (or any derivable range therein) fractionated doses are administered per day. In some embodiments, at least, at most, or exactly 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 (or any derivable range therein) fractionated doses are administered per week.

I. Surgery

Approximately 60% of persons with cancer will undergo surgery of some type, which includes preventative, diagnostic or staging, curative, and palliative surgery. Curative surgery includes resection in which all or part of cancerous tissue is physically removed, excised, and/or destroyed and may be used in conjunction with other therapies, such as the treatment of the present embodiments, chemotherapy, radiotherapy, hormonal therapy, gene therapy, immunotherapy, and/or alternative therapies. Tumor resection refers to physical removal of at least part of a tumor. In addition to tumor resection, treatment by surgery includes laser surgery, cryosurgery, electrosurgery, and microscopically-controlled surgery (Mohs' surgery).

Upon excision of part or all of cancerous cells, tissue, or tumor, a cavity may be formed in the body. Treatment may be accomplished by perfusion, direct injection, or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

J. Other Agents

It is contemplated that other agents may be used in combination with certain aspects of the present embodiments to improve the therapeutic efficacy of treatment. These additional agents include agents that affect the upregulation of cell surface receptors and GAP junctions, cytostatic and differentiation agents, inhibitors of cell adhesion, agents that increase the sensitivity of the hyperproliferative cells to apoptotic inducers, or other biological agents. Increases in intercellular signaling by elevating the number of GAP junctions would increase the anti-hyperproliferative effects on the neighboring hyperproliferative cell population. In other embodiments, cytostatic or differentiation agents can be used in combination with certain aspects of the present embodiments to improve the anti-hyperproliferative efficacy of the treatments. Inhibitors of cell adhesion are contemplated to improve the efficacy of the present embodiments. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with certain aspects of the present embodiments to improve the treatment efficacy.

K. Combination Cancer Therapies

The therapy provided herein may comprise administration of a combination of therapeutic agents, such as aAPCs, T cells, or NKT cells, and a second therapy. The therapies may be administered in any suitable manner known in the art. For example, the first and second cancer treatment may be administered sequentially (at different times) or concurrently (at the same time). In some embodiments, the first and second therapies are administered in a separate composition. In some embodiments, the first and second therapies are in the same composition. In some embodiments, methods and compositions of the disclosure comprise administration of an additional therapy. In some embodiments, the additional therapy comprises a cancer therapy such as an immunotherapy, a chemotherapy, radiation, or surgery.

Embodiments of the disclosure relate to compositions and methods comprising therapeutic compositions. The different therapies may be administered in one composition or in more than one composition, such as 2 compositions, 3 compositions, or 4 compositions. Various combinations of the agents may be employed, for example, a first cancer treatment is “A” and a second cancer treatment is “B”:

A/B/A B/A/B B/B/A A/A/B A/B/B B/A/A A/B/B/B B/A/B/B

B/B/B/A B/B/A/B A/A/B/B A/B/A/B A/B/B/A B/B/A/A

B/A/B/A B/A/A/B A/A/A/B B/A/A/A A/B/A/A A/A/B/A

The therapies comprising therapeutic agents such as polypeptides, nucleic acids, additional therapies, or aAPC therapies of the disclosure may be administered by the same route of administration or by different routes of administration. In some embodiments, the first therapy is administered intravenously, intramuscularly, subcutaneously, topically, orally, transdermally, intraperitoneally, intraorbitally, by implantation, by inhalation, intrathecally, intraventricularly, intratumoral, or intranasally. In some embodiments, the second therapy is administered intravenously, intramuscularly, subcutaneously, topically, orally, transdermally, intraperitoneally, intraorbitally, by implantation, by inhalation, intrathecally, intraventricularly, or intranasally. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined quantity of the therapeutic composition. The quantity to be administered, and the particular route and formulation, is within the skill of determination of those in the clinical arts. A unit dose need not be administered as a single injection but may comprise continuous infusion over a set period of time. In some embodiments, a unit dose comprises a single administrable dose.

The quantity to be administered, both according to number of treatments and unit dose, depends on the treatment effect desired. An effective dose is understood to refer to an amount necessary to achieve a particular effect. In the practice in certain embodiments, it is contemplated that doses in the range from 10 mg/kg to 200 mg/kg can affect the protective capability of these agents. Thus, it is contemplated that doses include doses of about 0.1, 0.5, 1, 5, 10, 15, 20, 25, 30, 35, 40, 45, 50, 55, 60, 65, 70, 75, 80, 85, 90, 100, 105, 110, 115, 120, 125, 130, 135, 140, 145, 150, 155, 160, 165, 170, 175, 180, 185, 190, 195, and 200, 300, 400, 500, 1000 μg/kg, mg/kg, μg/day, or mg/day or any range derivable therein. Furthermore, such doses can be administered at multiple times during a day, and/or on multiple days, weeks, or months.

In certain embodiments, the effective dose of the pharmaceutical composition is one which can provide a blood level of about 1 μM to 150 μM. In another embodiment, the effective dose provides a blood level of about 4 μM to 100 μM; or about 1 μM to 100 μM; or about 1 μM to 50 μM; or about 1 μM to 40 μM; or about 1 μM to 30 μM; or about 1 μM to 20 μM; or about 1 μM to 10 μM; or about 10 μM to 150 μM; or about 10 μM to 100 μM; or about 10 μM to 50 μM; or about 25 μM to 150 μM; or about 25 μM to 100 μM; or about 25 μM to 50 μM; or about 50 μM to 150 μM; or about 50 μM to 100 μM (or any range derivable therein). In other embodiments, the dose can provide the following blood level of the agent that results from a therapeutic agent being administered to a subject: about, at least about, or at most about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 μM or any range derivable therein. In certain embodiments, the therapeutic agent that is administered to a subject is metabolized in the body to a metabolized therapeutic agent, in which case the blood levels may refer to the amount of that agent. Alternatively, to the extent the therapeutic agent is not metabolized by a subject, the blood levels discussed herein may refer to the unmetabolized therapeutic agent.

Precise amounts of the therapeutic composition also depend on the judgment of the practitioner and are peculiar to each individual. Factors affecting dose include physical and clinical state of the patient, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units of μg/kg or mg/kg of body weight can be converted and expressed in comparable concentration units of μg/ml or mM (blood levels), such as 4 μM to 100 μM. It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

### VIII. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### A. Example 1-aAPC-Based Cancer Vaccines

In this example, the inventors demonstrate an approach to generate aAPC-based cancer vaccines that do not require identification and in vitro production of MHC-peptide complexes. This is a one-step process that allows the capture of the MHC-peptide complexes directly from the patient-derived tumor cell lysates to generate aAPCs. It is shown that the MHC I-peptide repertoire of normal- or tumor cells can be successfully captured directly from cell lysate using affinity beads. The aAPCs generated using this technique are able to induce antigen-specific cytotoxic effector T cell responses that led to in vitro and in vivo tumor cell killing.

1. Materials and Methods

Mice. OT-I Rag2−/− CD8 TCR transgenic mice specific for OVA257-264 (B6.129S6-Rag2tmlFwa Tg(TcraTcrb)1100Mjb) presented on H-2Kb and WT C57BL/6 mice were purchased from Taconic Biosciences (Rensselaer, N.Y.). All experiments were performed with 8 to 26-week-old mice. Mice were housed in microisolator cages and fed autoclaved food and acidified water. The Baylor Institutional Care and Use Committee approved all mouse protocols.

Cell lines. B16-OVA (B16F10 tOVA GFP, expressing truncated OVA and GFP) and parental B16F10 are a gift of Drs. Michael Gerner and Andrew Oberst (University of Washington). HEK293T cell line was purchased from ATCC (Manassas, Va.). Cells were cultured in Dulbecco's Modified Eagle Medium (Gibco, Grand Island, N.Y.) supplemented with 10% FBS, 1% Glutamax, 1% sodium pyruvate.

H-2Kb/OVA expression. The H-2Kb sequence was sub-cloned into cetHS-puro plasmid. As a result, Ctag sequence was fused to the C-terminus of the H-2Kb sequence. The successful generation of the construct was determined by PCR and sequencing (data not shown). One day prior to transfection the HEK293T cells were seeded in 10 cm tissue culture dish. By next day the cells reached 70%-80% confluence. At this time, the culture medium was replaced with 9 mL DMEM medium containing 25 μM chloroquine and the cells transfected with plasmids coding for Kb-Ctag and OVA (pcDNA3-OVA; Addgene, plasmid #64599). Briefly, 5 μg of Kb-Ctag and 5 μg OVA expressing plasmids were mixed in 450 μL H2O in 1.5 mL Eppendorf tube; 500 μL 2×HBSS was added sequentially. 50 μL 2 M CaCl₂ solution was then added and the tube was vortexed and kept on ice for 15 minutes. The plasmids were gently added on top of the cell cultures. For single transfections 10 μg of Kb-Ctag plasmid was used. On day 2 post transfection the cells were washed with warm DMEM medium twice and cultured for one extra day.

aAPC production. Kb-Ctag and OVA expressing 293 T cells (or Kb-Ctag expressing B16F10 cells) were lysed in lysis buffer (1% CHAPS, 25 mM Tris pH 7.5, 150 mM NaCl) containing protease inhibitor (cOmplete ULTRA™ Tablets; Roche, Mannheim, Germany). Lysis was performed at 4° C. for 1 hour. Supernatant was acquired by centrifuging the lysate at 12,000 rpm for 20 minutes. The cleared lysate was then mixed with Ctag matrix (CaptureSelect™ C-tag Affinity Matrix, Thermo Scientific, Waltham, Mass.), which has an aldehyde activated agarose matrix, and incubated at 4° C., on a slowly rotating surface for one hour. The matrix was then washed extensively with sterile PBS (500 rpm/20 seconds spin was used to recover the matrix). The successful pull-down of Kb:SIINFEKL pMHCI complex (or Kb) was determined by staining the matrix with antibodies that detect Kb and/or SIINFEKL bound to H-2Kb.

In vitro OT-I T cell activation. Secondary lymphoid organs from OT-I Rag1−/− mice were smashed through cell strainers and the red blood cells lysed using ACK. After washing, the cells were used as is or labeled with cell trace violet (CellTrace™ Violet Cell Proliferation Kit, Invitrogen, Carlsbad, Calif.) according to the manufacturer's instructions. The cells were then seeded in a 24-well plate in complete RPMI medium, each well containing 4 million cells in 2 mL medium. Control and experimental aAPCs (20 μL matrix for one well) were added to the cell cultures for 6 days. Every other day, half of the medium was replaced with fresh medium.

Flow cytometry. Staining was performed as previously described (Igyarto et al., 2011). Intracellular cytokine staining was performed with the BD Bioscience Cytofix/Cytoperm kit (BD Biosciences, San Jose, Calif.), according to the manufacturer's instructions. Samples were analyzed on LSRFortessa flow cytometer (BD Biosciences, San Jose, Calif.). The fluorochrome-conjugated antibodies to IFN gamma (XMG1.2), granzyme B (QA16A02), H-2Kb bound to SIINFEKL (25-D1.16), CD3ε (145-2C11), CD44 (IM7), CD90.1 (OX-7) and CD8a (3-6.7) were purchased from BioLegend (San Diego, Calif.). Anti-Kb antibody (Y-3) was purchased from BioXCell (West Lebanon, N.H.) and conjugated with Alexa Fluor™ 647 antibody labeling Kit (Invitrogen, Carlsbad, Calif.). Data were analyzed with FlowJo software (TreeStar; Ashland, Oreg.). All the flow cytometric plots displaying cells were pre-gated on live cells using Fixable Viability Dye eFluor 780 (eBioscience, San Diego, Calif.) and singlet events.

Adoptive transfer of OT-I T cells into tumor bearing mice. Eight-to-twelve weeks old WT C57BL/6 mice were inoculated subcutaneously with 106 B16-OVA cells. When the tumors became palpable (day 7) each mouse received through tail vein injection 4×10⁶ OT-I T cells stimulated in vitro with SIINFEKL, control or Kb/OVA aAPCs for 5 days. Female OT-I mice were used as T cell source. Tumors were measured using a caliper every three days and tumor volumes calculated based on the following formula: volume=(W²*L)/2, where W is width and L is length. As per approved animal protocol, the mice in which the tumor size has reached 1,000 mm³ or the animals showed distress such as visible weight loss, lack of grooming and feeding were euthanized.

Statistical analysis. Statistical analyses were performed using GraphPad Prism7.0 software (Graphpad, La Jolla, Calif.). Comparisons were made by Student's t-test and one-way ANOVA as noted in figure legends. A p value<0.05 was considered statistically significant.

2. Results

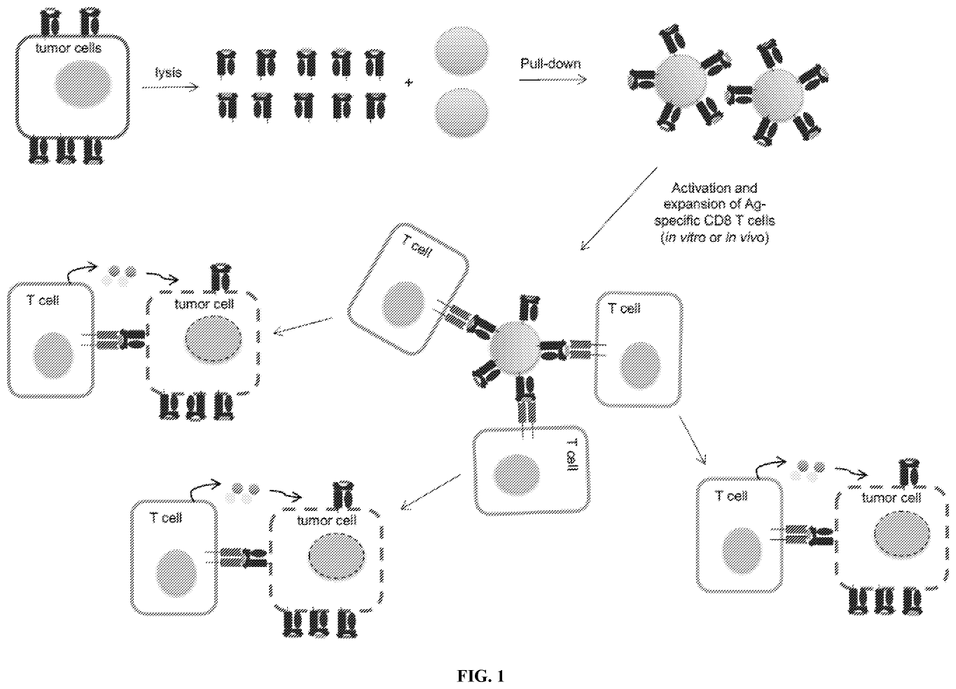

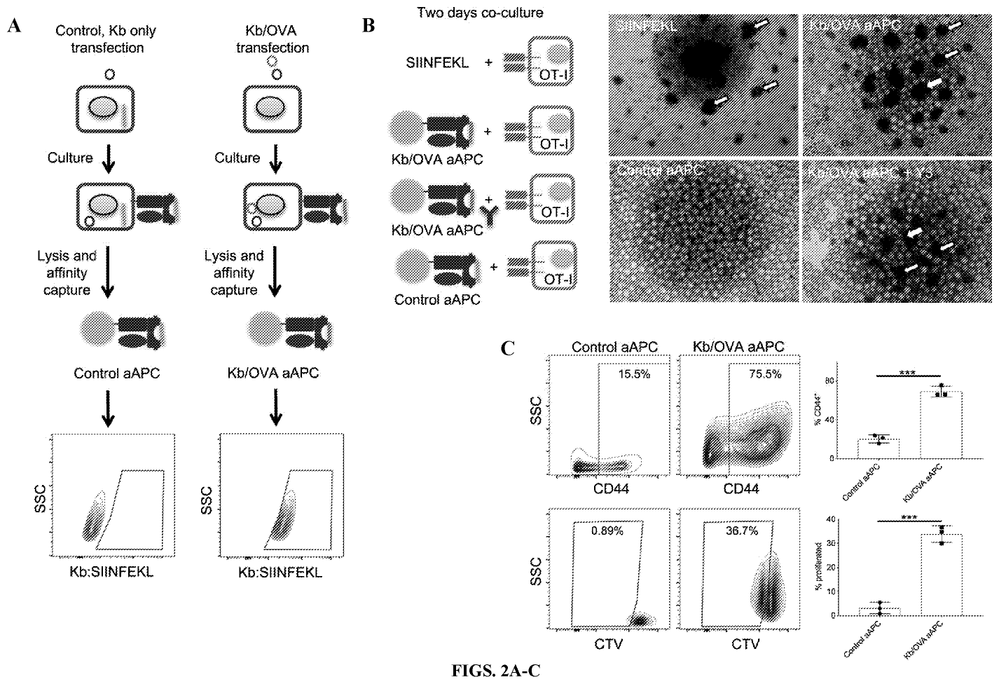

The concept behind one-step aAPCs. The production and wide use of aAPCs as cancer immunotherapeutics is hindered by the need to identify immunogenic cancer antigens and produce recombinant patient-specific HLAs loaded with these peptides. To overcome these limitations the inventors developed an approach wherein peptide-MHCs are directly captured from cell lysates, including of cancer cells, using affinity beads. The concept and strategy to generate one-step aAPCs are depicted in FIG. 1. The inventors designed a system to control and confirm each step of the aAPC generation. As a first step human embryonic kidney cells (HEK293T) were transfected with plasmids coding for C-tagged mouse H-2Kb (MHC-I) and OVA (FIG. 2A). The expression of the mouse H-2Kb on the cells surface was detected by Y 3 antibody (recognizes H-2Kb) and the formation of OVA-derived peptide MHC complexes with the use of antibody that recognizes SIINFEKL (dominant, OVA-derived CD8 epitope) bound to H-2Kb. Efficient expression of H-2Kb and loading of SIINFEKL peptide on MHC-I molecules was observed. No SIINFEKL staining was observed in the cells transfected only with Kb-Ctag plasmid. After confirming the expression of OVA-derived peptide in the context of MHC-I, the cells were lysed and the peptide-MHC-Is were purified using affinity matrix targeting the C-tag motif of the H-2Kb. The capture of MHC-I and MHC-I complexed with SIINFEKL were determined with the use of antibodies already presented above. The affinity beads efficiently captured MHC-I and MHC-I loaded with SIINFEKL (FIG. 2A). Thus, these data indicate that affinity beads targeting MHC-I could be used to generate patient-specific aAPCs.

aAPCs loaded with Kb:SIINFEKL can prime OT-I T cells. The successful generation of aAPCs using affinity matrix prompted the inventors to test whether the generated aAPCs can activate antigen-specific CD8 T cells. For this purpose, aAPCs were generated as presented above and were co-cultured with naïve OT-I cells labeled with CellTrace Violet (CTV). OT-I cells recognize SIINFEKL peptides presented in the context of H-2Kb. SIINFEKL peptide (2 μg/mL) stimulation served as a positive control (Pulle et al., 2006). The ex vivo culturing of OT-I T cells with aAPCs carrying Kb:SIINFEKL (Kb/OVA), but not control aAPCs (Kb), led to cell cluster formation (FIG. 2B). Inclusion of Y-3 antibody in cultures that binds to peptide groove of the H-2Kb, and thus interfere with TCR binding, decreased OT-I cluster formation. To confirm the activation and proliferation of OT-I cells, CTV-labeled OT-I cells were co-cultured with either control or Kb/OVA aAPCs and analyzed by flow cytometer. Kb/OVA aAPCs, unlike control aAPC, induced significant upregulation of CD44 and dilution of the proliferation dye by day 2 (FIG. 2C). AAPCs generated using the same technique but a different cell line (B16F10) provided similar results. Together, these data suggest that the aAPCs were able to prime antigen-specific T cell responses.

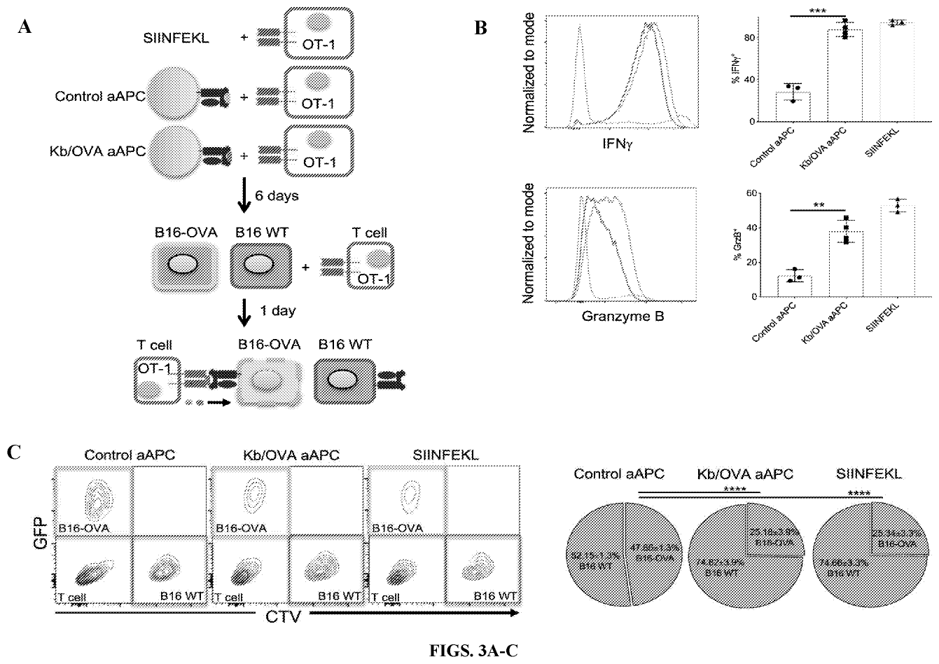

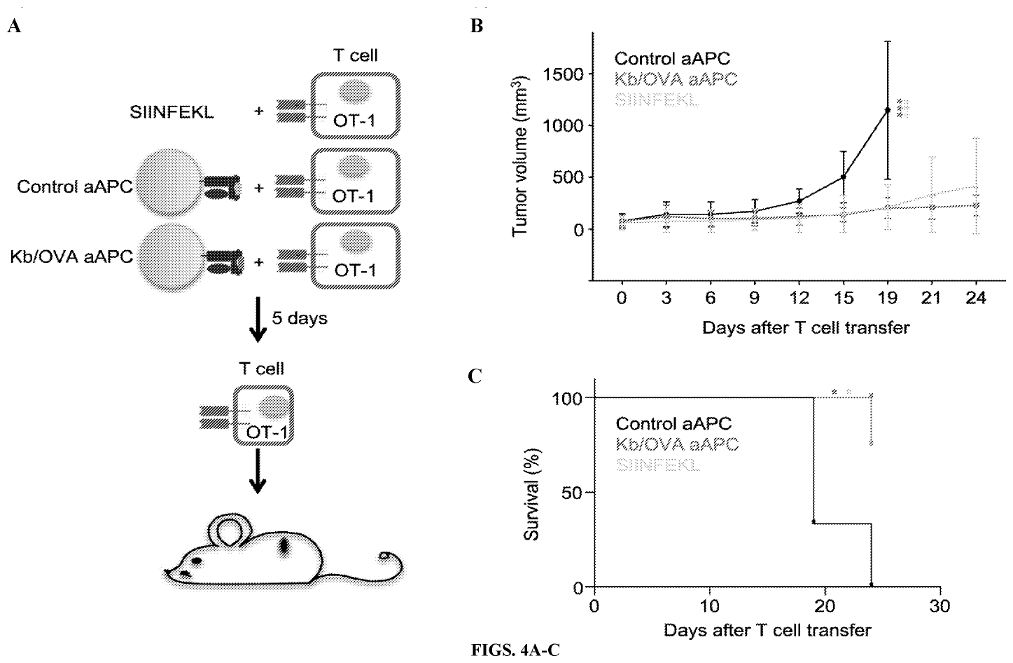

aAPC-activated T cells can kill tumor cells in vitro and in vivo. To investigate the cytotoxic potency of the aAPC-primed T cells, an in vitro tumor cell-killing assay using the B16F10 cell line was used. OT-I T cells were first primed by Kb: SIINFEKL aAPCs or control aAPCs for 6 days in vitro (FIG. 3A). Prior to use the cytotoxic phenotype (IFNγ and granzyme B) of the primed OT-I cells were confirmed by flow cytometry (FIG. 3B). B16F10 cells that express OVA and GFP (hereafter B16-OVA) were then mixed with their parental B16F10 cells that were labeled with CTV at a ratio of 1:1 (FIG. 3A). The primed OT-I T cells were added directly to B16 cell cultures one day after seeding at an effector to tumor cell ratio of 2:1. Peptide-stimulated OT-I cells served as positive controls. Cell counts were read by FACS one day after T cell addition. Both peptide- and aAPC-stimulated T cells effectively killed the tumor cells expressing OVA, but left the parental WT tumor cells intact (FIG. 3C). To further interrogate the cytotoxic ability of aAPC-stimulated T cells, the OT-I T cells were infused intravenously into WT mice carrying B16-OVA tumors (FIG. 4A). Both peptide and aAPC-stimulated OT-I cells significantly slowed the tumor growth (FIG. 4B) and increased survival (FIG. 4C). Collectively, the data indicated that this novel aAPC was able to induce cytotoxic T cells and could be used as immunotherapeutic.

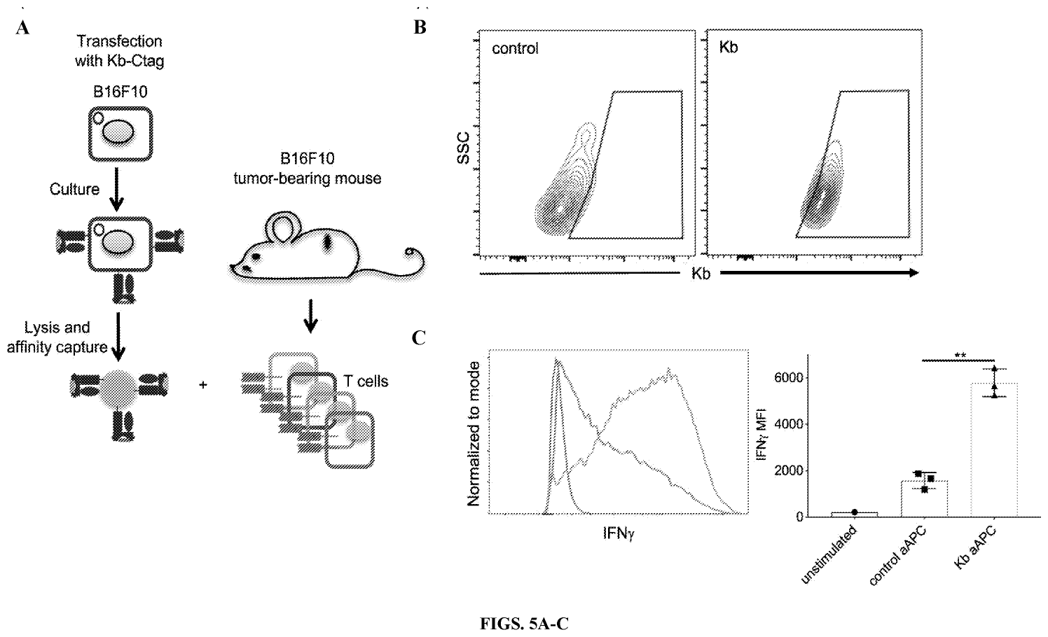

aAPCs generated using tumor cells were able to activate T cells from tumor-bearing mice. The inventors tested whether aAPCs generated by using unknown tumor antigens could be used to stimulate T cells isolated from tumor-bearing mice. To address this question, B16F10 cells were transfected with C-tagged H-2Kb and the peptide-MHC-Is were isolated as presented above. The scientific rationale behind this experiment was that these MHC-I molecules will be loaded with tumor antigens and the peptide-MHC-I repertoire of the B16F10 cells can be captured using affinity beads (FIG. 5A). FACS assay confirmed the successful capture of H-2Kbs from C-tagged H-2Kb transfected B16F10 cells (FIG. 5B). Next, splenocytes isolated from B16F10 tumor-bearing mice were isolated and stimulated with experimental and control aAPCs. The experimental aAPCs activated significantly higher numbers of CD8 T cells to produce IFNγ compared to control aAPCs (FIG. 5C), indicating that patient-specific aAPCs could be made.

Here the inventors present a novel and simple technique to generate patient-specific cancer vaccines. This experimental evidence can serve as the basis of the generation of patient-specific vaccines targeting cancer and autoimmune diseases. Affinity beads can be used to pull down peptide-MHC directly from cell lysates, and it is a viable option to generate patient-specific aAPCs in matters of days at minimal costs.

The clinical usability of aAPCs generated until now to treat cancer were limited by multiple factors. AAPCs have to be patients specific that first require characterization of the patient's HLA haplotype and identification of cancer neoantigens. The HLA haplotype is fairly easily determined by PCR and flow cytometry, however identification of immunogenic cancer neoantigens is a very expensive, tedious, and labor-expensive process with many caveats along the pipeline that further limit its success rate (Ebstein et al., 2016; Hundal et al., 2016; Laumont et al., 2016; Liepe et al., 2016). Since most of the cancers are highly heterogeneous a dominant neoantigen might only target some of the cancer cells, and the neoantigen could be patient-specific or it will only bind to certain HLA molecules, limiting their wider usability. To make the aAPCs, the correct HLAs have to be produced as recombinant proteins and assembled with the corresponding neoantigenic peptides. The recombinant HLA and peptides might not carry all the posttranslational modifications that would normally occur in vivo that could result in less effective TCR stimulation. Furthermore, the whole spectrum of HLA is hard to reconstruct. The inventors addressed these problems, demonstrating successful pull-down of peptide-MHCs that can later be used to stimulate antigen-specific anti-cancer effector T cell responses. Tumor-specific T cell clones are of low abundance in nature (Scheper et al., 2019), and unlike most of the aAPCs generated to date, which often only present one antigenic peptide, this technique, by capturing a diverse peptide-MHC-I pool increases the chance to target and activate multiple cancer-specific T cell clones. It is expected that the peptide repertoire presented by the cancer cells and the tumor heterogeneity will be represented proportionally on the aAPCs. If later studies prove otherwise, a modified version of the Drop-sequencing technology can be used, where in this case a lipid droplet containing lysis buffer will form around one cancer cell and one affinity bead. This will assure that every aAPC will represent one cancer cell's peptide-MHC-I repertoire. Tumor samples with higher leukocytic infiltrates might require purification steps to enrich for tumor-derived signatures. In some instances tumor cells escape immune surveillance by downregulating surface expression of MHC-I (Bubenik, 2003). In theory, aAPCs disclosed herein could still capture the intracellularly retained peptide-MHC-I repertoire; or as a last resort, ectopic transfection of tumor cells with patient-specific HLA-I could circumvent this caveat. The aAPCs generated using this technique will probably contain, self, non-mutated, non-immunogenic peptides that could trigger some sorts of autoimmune responses if combined with co-stimulation. This however, is expected to be minimal, because of central and peripheral tolerance. The possible autoimmune symptoms could also be controlled by the use of different drugs.

Because of lack of access to antibodies that would recognize the cytoplasmic part of mouse H-2Kb and for versatility reasons, in these in vitro and in vivo experiments, tag-specific antibodies were used. Antibodies that recognize cytoplasmic portion of the Kb or target beta-2 microglobulin could also be used to capture the peptide-MHC-I complexes from C57BL/6 mouse samples. For human studies and for the generation of human aAPCs the W6/32 pan HLA-I antibody could be used, for example. Viral proteins that interact with the cytoplasmic portion of HLA-I could be also a viable option for affinity purification of peptide-MHC-I repertoire and generation of aAPCs.

In some embodiments, the aAPC generation technique disclosed is applied to achieve both immunogenic and tolerogenic immune responses. In such embodiments, aAPCs are generated from normal cells or cells pulsed with self-antigen and combined with inhibitory signals and cytokine, leading to generation of antigen-specific regulatory T cell responses (prevent or treat autoimmune diseases). Along the same logic aAPCs can be generated to treat allergy or used as preventative vaccines to fight infectious diseases.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Bubenik, J. (2003). Tumour MHC class I downregulation and immunotherapy (Review). Oncol Rep 10, 2005-2008.
Chaput, N., Schartz, N. E., Andre, F., and Zitvogel, L. (2003). Exosomes for immunotherapy of cancer. Adv Exp Med Biol 532, 215-221.
Ebstein, F., Textoris-Taube, K., Keller, C., Golnik, R., Vigneron, N., Van den Eynde, B. J., Schuler-Thurner, B., Schadendorf, D., Lorenz, F. K., Uckert, W., et al. (2016). Proteasomes generate spliced epitopes by two different mechanisms and as efficiently as non-spliced epitopes. Sci Rep 6, 24032.
Fu, C., and Jiang, A. (2018). Dendritic Cells and CD8 T Cell Immunity in Tumor Microenvironment. Front Immunol 9, 3059.
Han, H., Peng, J. R., Chen, P. C., Gong, L., Qiao, S. S., Wang, W. Z., Cui, Z. Q., Yu, X., Wei, Y. H., and Leng, X. S. (2011). A novel system of artificial antigen-presenting cells efficiently stimulates Flu peptide-specific cytotoxic T cells in vitro. Biochem Biophys Res Commun 411, 530-535.
Hundal, J., Carreno, B. M., Petti, A. A., Linette, G. P., Griffith, O. L., Mardis, E. R., and Griffith, M. (2016). pVAC-Seq: A genome-guided in silico approach to identifying tumor neoantigens. Genome Med 8, 11.
Laport, G. G., Levine, B. L., Stadtmauer, E. A., Schuster, S. J., Luger, S. M., Grupp, S., Bunin, N., Strobl, F. J., Cotte, J., Zheng, Z., et al. (2003). Adoptive transfer of costimulated T cells induces lymphocytosis in patients with relapsed/refractory non-Hodgkin lymphoma following CD34+-selected hematopoietic cell transplantation. Blood 102, 2004-2013.
Laumont, C. M., Daouda, T., Laverdure, J. P., Bonneil, E., Caron-Lizotte, O., Hardy, M. P., Granados, D. P., Durette, C., Lemieux, S., Thibault, P., et al. (2016). Global proteogenomic analysis of human MHC class I-associated peptides derived from non-canonical reading frames. Nat Commun 7, 10238.
Liepe, J., Marino, F., Sidney, J., Jeko, A., Bunting, D. E., Sette, A., Kloetzel, P. M., Stumpf, M. P., Heck, A. J., and Mishto, M. (2016). A large fraction of HLA class I ligands are proteasome-generated spliced peptides. Science 354, 354-358.
Lu, X., Jiang, X., Liu, R., Zhao, H., and Liang, Z. (2008). Adoptive transfer of pTRP2-specific CTLs expanding by bead-based artificial antigen-presenting cells mediates anti-melanoma response. Cancer Lett 271, 129-139.
Merad, M., Sathe, P., Helft, J., Miller, J., and Mortha, A. (2013). The dendritic cell lineage: ontogeny and function of dendritic cells and their subsets in the steady state and the inflamed setting. Annu Rev Immunol 31, 563-604.
Mitchell, M. S., Darrah, D., Yeung, D., Halpern, S., Wallace, A., Voland, J., Jones, V., and Kan-Mitchell, J. (2002). Phase I trial of adoptive immunotherapy with cytolytic T lymphocytes immunized against a tyrosinase epitope. J Clin Oncol 20, 1075-1086.
Oelke, M., Maus, M. V., Didiano, D., June, C. H., Mackensen, A., and Schneck, J. P. (2003). Ex vivo induction and expansion of antigen-specific cytotoxic T cells by HLA-Ig-coated artificial antigen-presenting cells. Nat Med 9, 619-624.
Palucka, K., and Banchereau, J. (2013). Dendritic-cell-based therapeutic cancer vaccines. Immunity 39, 38-48.
Pulle, G., Vidric, M., and Watts, T. H. (2006). IL-15-dependent induction of 4-1BB promotes antigen-independent CD8 memory T cell survival. J Immunol 176, 2739-2748.
Reddy, S. T., Rehor, A., Schmoekel, H. G., Hubbell, J. A., and Swartz, M. A. (2006). In vivo targeting of dendritic cells in lymph nodes with poly(propylene sulfide) nanoparticles. J Control Release 112, 26-34.
Sallusto, F., and Lanzavecchia, A. (2002). The instructive role of dendritic cells on T-cell responses. Arthritis Res 4 Suppl 3, S127-132.
Scheper, W., Kelderman, S., Fanchi, L. F., Linnemann, C., Bendle, G., de Rooij, M. A. J., Hirt, C., Mezzadra, R., Slagter, M., Dijkstra, K., et al. (2019). Low and variable tumor reactivity of the intratumoral TCR repertoire in human cancers. Nat Med 25, 89-94.
Shao, J., Xu, Q., Su, S., Wei, J., Meng, F., Chen, F., Zhao, Y., Du, J., Zou, Z., Qian, X., et al. (2018). Artificial antigen-presenting cells are superior to dendritic cells at inducing antigen-specific cytotoxic T lymphocytes. Cell Immunol 334, 78-86.
Steenblock, E. R., and Fahmy, T. M. (2008). A comprehensive platform for ex vivo T-cell expansion based on biodegradable polymeric artificial antigen-presenting cells. Mol Ther 16, 765-772.
Ugel, S., Zoso, A., De Santo, C., Li, Y., Marigo, I., Zanovello, P., Scarselli, E., Cipriani, B., Oelke, M., Schneck, J. P., et al. (2009). In vivo administration of artificial antigen-presenting cells activates low-avidity T cells for treatment of cancer. Cancer Res 69, 9376-9384.
Wang, C., Sun, W., Ye, Y., Bomba, H. N., and Gu, Z. (2017). Bioengineering of Artificial Antigen Presenting Cells and Lymphoid Organs. Theranostics 7, 3504-3516.
Jiang, et al., Adv. Drug Deliv. Rev., 57(3):391-410.
Aguado and Lambert, Immunobiology, 184(2-3):113-25 (1992).
Bramwell, et al., Adv. Drug Deliv. Rev., 57(9):1247-65 (2005).
Lanzavecchia, Curr. Opin. Immunol., 8:348-54 (1996).
Wick, et al., Immunol. Rev., 172:153-62 (1999).
Lehner, et al., Curr. Biol., 8: R605-8 (1998).
Braciale, Curr. Opin. Immunol., 4:59-62 (1992).
Fyfe, et al., J. Clin. Oncol., 13(3):688-96 (1995).
Papadopoulos, et al., N. Engl. J. Med, 330(17):1185-91 (1994).
Savoldo, et al., Leuk Lymphoma, 39(5-6):455-64 (2000).
Hori, et al., Adv. Immunol., 81:331-71 (2003).
Karim, et al., J. Immunol., 172(2):923-8 (2004).
Dudley, et al., Nat. Rev. Cancer, 3(9):666-75 (2003).
Riddell, et al., Cancer Control, 9(2):114-22 (2002).
Yee, et al., Proc. Natl. Acad. Sci. USA., 99(25):16168-73 (2002).
Michel, et al., Immunity, 15(6):935-45 (2001).
Pardoll, Nat. Rev. Immunol., 2(4):227-38 (2002).
Fyfe, et al., J. Clin. Oncol., 13(3):688-96 (1995).
Schluns, et al., Nat. Rev. Immunol, 3(4):269-79 (2003).
Coligan et al., Current Protocols in Immunology, John Wiley & Sons Inc. (1994).
Rocha, et al., Science, 251:1225-1228 (1991).
Kisielow, et al., Nature, 333:742-746 (1988).
Schwartz, Science, 248:1349-1356 (1990).
Miller, et al., Immunol. Rev., 133:131-150 (1993).
Sakaguchi, et al., J. Immunol., 155:1151-1164 (1995).
Takahashi, et al., Int. Immunol., 10:1969-1980 (1998).
Itoh, et al., J. Immunol., 162:5317-5326 (1999).
Shimizu, et al., J. Immunol., 163:5211-5218 (1999).
King et al., Front. Immunol., 9:1519 (2018).
Nair & Dhodapkar, Front. Immunol., 8:1178 (2017).
US2014/0294898
US2014/022021
US2011/0008369
U.S. Pat. No. 6,207,156
U.S. Pat. No. 8,735,553
U.S. Pat. No. 8,354,509
U.S. Pat. No. 8,008,449
U.S. Pat. No. 8,119,129
U.S. Pat. No. 8,017,114
WO2009/114335
WO2009/101611
WO2010/027827
WO2011/066342
WO2001/14424
WO1998/42752
WO2000/37504
WO2001/014424
WO2000/037504

What is claimed is:

1. A method of producing activated antigen-specific cytotoxic T cells, the method comprising contacting CD8+ T cells with a composition comprising substrate particles bound to endogenous MHC I-peptide complexes obtained from one or more cells.

2. The method of claim 1, wherein the endogenous MHC I-peptide complexes comprise a plurality of different MHC I-peptide complexes.

3. The method of claim 2, wherein the plurality of different endogenous MHC I-peptide complexes represents a repertoire of the MHC I-peptide complexes expressed by the one or more cells.

4. The method of any one of claims 1 to 3, wherein the endogenous MHC I-peptide complexes comprise peptides from one or more tumor-specific antigens.

5. The method of any one of claims 1 to 4, wherein the endogenous MHC I-peptide complexes are bound to the substrate particles via a polypeptide capable of specifically binding MHC.

6. The method of any one of claims 1 to 5, wherein the substrate particles are not bound to recombinantly expressed MHC complexes.

7. The method of any one of claims 1 to 6, wherein the endogenous MHC I-peptide complexes are obtained from a lysate prepared from the one or more cells.

8. The method of claim 7, further comprising coupling the substrate particles to the endogenous MHC I-peptide complexes obtained from the one or more cells by a method comprising: contacting a lysate of the one or more cells with substrate particles bound to a polypeptide capable of specifically binding MHC, wherein the lysate of the one or more cells contains endogenous MHC I-peptide complexes produced by the one or more cells.

9. The method of any one of claims 1 to 8, wherein the substrate particles are additionally bound to one or more of the following T cell co-stimulatory molecules: CD80, CD86, OX-40L, 4-1BBL, CD70, ICOS-L, and GITR-L, or any combination thereof.

10. The method of any one of claims 1 to 9, wherein the substrate particles are additionally bound to one or more polypeptides that specifically bind and activate one or more of the following T cell molecules: CD28, OX40, 4-1BB, CD27, ICOS, and GITR, or any combination thereof.

11. The method of any one of claims 1 to 10, wherein the substrate particles further comprise a T cell activating cytokine.

12. The method of claim 11, wherein the T cell activating cytokine is released and stimulates the CD8+ T cells.

13. The method of claim 11 or 12, wherein the T cell activating cytokine is selected from IL-2, IL-7, IL-12, IL-15, or IL-21, or any combination thereof.

14. The method of any one of claims 1 to 13, wherein the substrate particles further comprise a T cell chemoattractant selected from CXCL9, CXCL10, CXCL12 CXCL16, CCL3, CCL4, CCL19, or CCL21, or any combination thereof.

15. The method of any one of claims 1 to 14, wherein the substrate particles comprise a polymer material, a magnetic material, or a lipid bilayer.

16. The method of any one of claims 1 to 15, wherein the substrate particles are about 1 to 20 μm in size.

17. The method of any one of claims 1 to 16, wherein the substrate particles are biodegradable.

18. The method of any one of claims 1 to 17, wherein the endogenous MHC I-peptide complexes are obtained from a single cell.

19. The method of claim 18, further comprising coupling the substrate particles to the endogenous MHC I-peptide complexes obtained from a single cell by a method comprising: contacting a lysate prepared from the single cell with substrate particles bound to a polypeptide capable of specifically binding MHC, wherein the lysate contains endogenous MHC I-peptide complexes produced by the cell. In some embodiments, the single cell is comprised within a continuous lipid membrane with a single substrate particle.

20. The method of any one of claims 1 to 19, wherein contacting the CD8+ T cells with the composition occurs in vivo.

21. The method of claim 20, wherein the CD8+ T cells are within a patient's body at the time of the contacting.

22. The method of claim 20 or 21, further comprising administering the composition to the patient.

23. The method of any one of claims 1 to 19, wherein contacting the CD8+ T cells with the composition occurs ex vivo.

24. The method of claim 23, wherein the CD8+ T cells have been removed from a patient before the contacting.

25. The method of claim 24, further comprising administering the CD8+ T cells to the patient after the contacting.

26. The method of any one of claims 1 to 25, wherein the contacting causes activation and proliferation of CD8+ T cells specific for one or more MHC I-peptide complexes present in the MHC I-peptide complexes bound to the substrate particles.

27. The method of any one of claims 1 to 26, wherein the one or more cells comprise one or more cancer cells obtained from a patient.

28. The method of claim 27, wherein the contacting causes activation and proliferation of CD8+ T cells capable of killing cancer cells in the patient.

29. The method of claim 27 or 28, wherein the one or more cancer cells obtained from the patient comprise a tumor biopsy.

30. The method of claim 29, wherein the tumor biopsy comprises a heterogeneous population of cancer cells.

31. A method of killing a target cell, the method comprising:
(a) contacting CD8+ T cells with a with a composition comprising substrate particles bound to endogenous MHC I-peptide complexes obtained from one or more cells of the same type as the target cell, thereby creating activated, target antigen-specific CD8+ T cells; and
(b) contacting the target cell with the activated, target antigen-specific CD8+ T cells, thereby killing the target cell.

32. The method of claim 31, wherein steps (a) and (b) occur in vivo.

33. The method of claim 31 or 32, wherein step (a) comprises administering the composition to a patient to cause the contacting.

34. The method of any one of claims 31 to 33, wherein the endogenous MHC I-peptide complexes are obtained from a lysate prepared from the one or more cells.

35. The method of any one of claims 31 to 34, wherein the endogenous MHC I-peptide complexes comprise a plurality of different MHC I-peptide complexes.

36. A method of treating a condition in a patient comprising administering to the patient a composition comprising substrate particles bound to endogenous MHC I-peptide complexes obtained from one or more cells of the same type as a target cell, wherein the target cell is killed, and wherein killing the target cell treats the condition.

37. The method of claim 36, wherein the condition is cancer.

38. The method of claim 36 or 37, wherein the one or more cells of the same type as the target cell were obtained from the patient.

39. The method of any one of claims 36 to 38, wherein the target cell is a tumor cell and the one or more cells of the same type as the target cell were obtained from the same tumor in which the target cell was present before being killed.

40. A method of treating cancer in a patient comprising:
(a) preparing a solution comprising endogenous MHC I-peptide complexes obtained from one or more cancer cells;
(b) contacting the solution with substrate particles bound to a polypeptide that specifically binds MHC to pull down the endogenous MHC I-peptide complexes; and
(c) administering the substrate particles produced in step (b) to the patient.

41. The method of claim 40, wherein the solution is a lysate of the one or more cancer cells.

42. The method of claim 41, wherein the lysate is prepared from a single cancer cell.

43. The method of claim 41, wherein the lysate is prepared from a tissue sample obtained from the patient.

44. A method of making an artificial antigen presenting cell, the method comprising contacting a lysate prepared from one or more cells with substrate particles bound to a polypeptide that is capable of specifically binding MHC, thereby causing the substrate particles to become bound to a plurality of different endogenous MHC I-peptide complexes expressed by the one or more cells.

45. The method of claim 44, wherein the substrate particles are further bound to one or more of the following T-cell co-stimulatory molecules: CD80, CD86, OX-40L, 4-1BBL, CD70, ICOS-L, and GITR-L, or any combination thereof.

46. The method of claim 44 or 45, wherein the substrate particles are further bound to one or more polypeptides that specifically bind and activate one or more of the following T cell molecules: CD28, OX40, 4-1BB, CD27, ICOS, and GITR, or any combination thereof.

47. The method of any one of claims 44 to 46, wherein the substrate particles comprise a polymer material, a magnetic material, or a lipid bilayer.

48. The method of any one of claims 44 to 47, wherein the substrate particles are biodegradable.

49. The method of any one of claims 44 to 48, wherein the lysate is prepared from a single cell.

50. A composition comprising substrate particles bound to:
(a) a plurality of different endogenous MHC I-peptide complexes, wherein the MHC I-peptide complexes are bound to the substrate particles via a polypeptide that specifically binds MHC; and
(b) one or more of the following molecules:
(i) a T-cell costimulatory molecule selected from CD80, CD86, OX-40L, 4-1BBL, CD70, ICOS-L, and GITR-L; or
(ii) one or more polypeptides that specifically bind and activate one or more of the following T cell molecules: CD28, OX40, 4-1BB, CD27, ICOS, and GITR.

51. The composition of claim 50, wherein the substrate particles further comprise a signaling molecule that can be released from the substrate particles and modulate T cell activity.

52. The composition of claim 50 or 51, wherein the substrate particles comprise a polymer material, a magnetic material, or a lipid bilayer.

53. The composition of any one of claims 50 to 52, wherein the substrate particles are biodegradable.

54. The composition of any one of claims 50 to 53, wherein the plurality of different endogenous MHC I-peptide complexes represents a repertoire of MHC I-peptide complexes expressed by one or more cells.

55. A method of inducing immune tolerance to an antigen in a subject, the method comprising:
(a) obtaining artificial antigen presenting cells comprising substrate particles bound to:
(i) endogenous MHC II complexes bound to peptides of the antigen, wherein the endogenous MHC II complexes were obtained from one or more cells containing the autoantigen; and
(ii) one or more inhibitory ligands selected from B7.1wa, CTLA-4 binding proteins, PD-L1, HVEM, PDL-2, B7-H3, B7-H4, OX-2, TGF-beta1, IL-10, IL-4, natural, recombinant and artificial ligands for CTLA-4, PD-1, OX-2 receptor, B7-H3 receptor, B7-H4 receptor and BTLA, natural and recombinant anti-CTLA-4 agonistic antibody, anti-PD-1 agonistic antibody, anti BTLA agonistic Antibody Anti-B7-H3 receptor agonistic antibody, Anti-B7-H4 receptor antibody, anti-CD28 antagonistic antibody, and anti-ICOS antagonistic antibody.
(b) administering a pharmaceutical composition comprising the artificial antigen presenting cells to the subject.

56. A method of inducing immune tolerance to an antigen in a subject, the method comprising:
(a) obtaining artificial antigen presenting cells comprising substrate particles bound to:
(i) endogenous MHC II complexes bound to peptides of the antigen, wherein the endogenous MHC II complexes were obtained from one or more cells containing the autoantigen; and
(ii) one or more inhibitory ligands selected from B7.1wa, CTLA-4 binding proteins, PD-L1, HVEM, PDL-2, B7-H3, B7-H4, OX-2, TGF-beta1, IL-10, IL-4, natural, recombinant and artificial ligands for CTLA-4, PD-1, OX-2 receptor, B7-H3 receptor, B7-H4 receptor and BTLA, natural and recombinant anti-CTLA-4 agonistic antibody, anti-PD-1 agonistic antibody, anti BTLA agonistic Antibody Anti-B7-H3 receptor agonistic antibody, Anti-B7-H4 receptor antibody, anti-CD28 antagonistic antibody, and anti-ICOS antagonistic antibody;
(b) ex vivo, contacting regulatory T cells specific for the antigen with the artificial antigen presenting cells; and
(c) administering the regulatory T cells to the subject.

57. A method of treating a condition in a patient, the method comprising:
(a) obtaining cells from the patient;
(b) preparing a lysate from the cells, wherein the lysate comprises endogenous MHC-peptide complexes produced by the cells;
(c) contacting the lysate with substrate particles bound to an anti-MHC antibody or functional fragment thereof, thereby generating aAPCs comprising the substrate particles bound to the endogenous MHC-peptide complexes;
(d) administering the aAPCs to the patient.

58. A method of treating a condition in a patient, the method comprising administering aAPCs to the patient, wherein the aAPCs comprise substrate particles bound to endogenous MHC-peptide complexes, wherein the aAPCs have been prepared by contacting a lysate prepared from cells obtained from the patient with the substrate particles, and wherein the substrate particles are bound to a polypeptide capable of specifically binding endogenous MHC I-peptide complexes in the lysate.
